(19) United States
(12) Patent Application Publication
(10) Pub. No.: US 2026/0327560 A1
(43) Pub. Date: Oct. 22, 2026

(54) METHODS FOR EX VIVO EXPANSION OF TUMOR INFILTRATING LYMPHOCYTES

(71) Applicant: MENDUS B.V., Leiden (NL)

(72) Inventors: Erik Hans Manting, Leiden (NL); Alex Karlsson-Parra, Leiden (NL); Satwinder Kaur Singh, Leiden (NL); Haoxiao Zuo, Leiden (NL)

(21) Appl. No.: 19/454,122

(22) Filed: Jan. 20, 2026

Related U.S. Application Data
(60) Provisional application No. 63/747,755, filed on Jan. 21, 2025.
(60) Provisional application No. 63/909,217, filed on Oct. 31, 2025.

(51) Int. Cl. C12N 5/0783

(52) U.S. Cl. CPC C12N 5/0636; C12N 2501/2302; C12N 2501/515; C12N 2502/1121

(57) ABSTRACT

The present disclosure provides methods which employ modified cells of leukemic origin for ex vivo expansion of tumor infiltrating lymphocytes (TILs) for use in adoptive cell therapy.

### CROSS REFERENCE TO RELATED APPLICATIONS

The present patent application claims the priority benefit of U.S. Provisional Patent Application Ser. Nos. 63/747,755, filed Jan. 21, 2025 and 63/909,217, filed Oct. 31, 2025, the contents of which are hereby incorporated by reference in their entirety into this disclosure.

### BACKGROUND

Tumor-infiltrating lymphocytes (TILs) represent a heterogeneous population of lymphocytes that grow within a tumor. This population as a whole is ineffective in eradicating the tumor in which they grow for complex reasons that likely include the presence of an inadequate numbers of anti-tumor cells, the presence of anti-tumor cells that have become senescent or anergic, and high numbers of immunosuppressive cells present in the tumor, such as regulatory T cells. Local secreted factors by myeloid-derived suppressor cells also contribute to tumor immunosuppression in the tumor microenvironment.

The rationale of TIL therapy is that an anti-tumor immune response can be enhanced by removing cells with anti-tumor potential from the immunosuppressive tumor microenvironment to a setting where they can be expanded in vitro and then returned in high enough numbers that allow them to traffic to tumor sites and kill tumor targets and possibly other cell targets that sustain the tumor, such as vascular endothelial cells (Lee et al, Curr Oncol Rep. 2012 October; 14(5): 468-474). Moreover, a response to TIL therapy not only requires transfer of cells with immediate cytolytic effector function to kill the bulk of fast-growing tumor, but also requires transfer of tumor-specific cells that maintain an ability for self-renewal and the capacity to produce a continual supply of cytolytic effector progeny until all malignant cells are eliminated.

TIL therapy shows great promise in the treatment of patients with advanced malignant melanoma. (Chesney et al., Journal for immunotherapy of cancer 10, no. 12(2022 ).) However, the translation of TIL therapy to non-melanoma solid tumors has been less successful with a major constraint being the inability to reproducibly generate enough quantity of TILs from primary and metastatic tumor tissues.

Fresh TILs are usually highly differentiated, and the standard protocol for expanding TILs to therapeutic levels is known to drive a substantial number of expanded TILs to become terminally differentiated and further exhausted. (Parkhurst et al., Nature Medicine 30, no. 9 (2024): 2586-2595; Cromton et al, Immunological reviews, 257, no. 1(2014 ), 264-276). New strategies for expanding TILs to therapeutic levels while addressing the problem of terminal differentiation of expanded TILs are desired.

### SUMMARY

The present disclosure is based on the discovery that addition of a leukemic cell derived mature dendritic cells to standard pre-rapid expansion (pre-REP) protocols can increase the IL-2-dependent proliferation of TILs during the pre-REP phase without negatively affecting the subsequent rapid expansion of TILs using the standard rapid expansion protocol (REP), while concomitantly avoiding increased terminal differentiation and exhaustion of the expanded cells.

In one aspect, the disclosure is a method for stimulation and expansion of TILs, the method comprising: (a) performing a first phase expansion (i.e., pre-REP expansion, or pre-rapid expansion) of TILs by co-culturing a first population of TILs from a tumor sample and a population of modified cells of leukemic origin in a pre-rapid expansion protocol (pre-REP) culture comprising interleukin-2 (IL-2), thereby producing a second population of TILs, wherein the population of modified cells of leukemic origin comprises a mature dendritic cell phenotype and is non-proliferating, and wherein the second population of TILs is greater in number than the first population of TILs; and (b) performing a second phase of expansion (i.e., a REP expansion, or rapid expansion) by culturing the second population of TILs in a rapid expansion protocol (REP) culture comprising an anti-CD3 antibody, at least one cytokine, and irradiated allogeneic peripheral blood mononuclear cells (PBMCs) without addition of the population of modified cells of leukemic origin, thereby producing a third population of TILs, wherein the third population of TILs is greater in number than the second population of TILs.

In various embodiments, the third population of TILs comprises a decreased subpopulation of terminally differentiated effector memory T cells (T_{EMRA}) despite an increase of the frequency of differentiated effector-memory (EM) phenotype relative to a population of TILs produced by a process including a pre-REP expansion and a subsequent REP expansion in which the pre-REP expansion is performed without addition of the population of modified cells of leukemic origin.

In certain embodiments, the third population of TILs exhibits a lower grade of exhaustion relative to a population of TILs produced by a process including a pre-REP expansion and a subsequent REP expansion in which the pre-REP expansion is performed without addition of the population of modified cells of leukemic origin.

In certain embodiments, the method results in a greater expansion of TILs from the second population of TILs to the third population of TILs during the REP phase, relative to a process including a pre-REP expansion and a subsequent REP expansion in which the pre-REP expansion is performed without addition of the population of modified cells of leukemic origin.

In certain exemplary embodiments, the third population of TILs is at least 1,000-fold greater in number than the second TIL population when the pre-REP expansion is performed for about 2 weeks and the REP expansion is performed for about 2 weeks.

In certain exemplary embodiments, the tumor sample is obtained from a tumor tissue of a subject having cancer, wherein the tumor tissue is from surgical resection, needle biopsy, core biopsy, small biopsy, or other means from a subject having cancer, and wherein the tumor sample contains a mixture of tumor cells and the first population of TILs.

In certain exemplary embodiments, the method described above further comprises a step of enzymatically digesting the tumor tissue prior to performing the first phase expansion, optionally wherein the tumor tissue is enzymatically digested by an enzyme chosen from collagenases, DNAse, and/or hyaluronidase.

In certain exemplary embodiments, the first population of TILs is a population of unselected TILs comprised in the tumor sample, and wherein the tumor sample is co-cultured with the population of modified cells of leukemic origin in the pre-REP culture.

In certain exemplary embodiments, the tumor sample is from melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma. In certain other exemplary embodiments, the tumor sample is from ovarian cancer or endometrial cancer.

In certain exemplary embodiments, the first phase expansion is performed for a period of about 7 days to about 21 days (e.g., 7, 8, 9, 10, 11, 12, 13, 14, 15, 16, 17, 18, 19, 20 or 21 days). In one embodiment, the first phase expansion is performed for about 14 days. In another embodiment, the first phase expansion is performed for about 21 days.

In certain exemplary embodiments the ratio of the number of the modified cells of leukemic origin added in the first phase expansion culture to the number of TILs in the first population is from about 1:10 to about 1:1.

In certain exemplary embodiments, IL-2 is present in the pre-REP phase culture at a concentration ranging from about 1000 IU/mL to about 6000 IU/mL. In one embodiment, IL-2 is present in the culture during the pre-REP phase at a concentration of about 6000 IU/mL.

In certain exemplary embodiments, the second population of TILs is greater in number than a population of TILs produced by culturing the first population of TILs in a pre-REP phase without addition of the population of modified cells of leukemic origin. In one exemplary embodiment, TILs are expanded at least 5-fold (e.g., 5-fold, 7.5-fold, 10-fold, 15-fold or 20-fold) at the completion of the first expansion in the pre-REP phase.

In certain exemplary embodiments, the subpopulation of T cells constitutes at least 60% of the total second population of TILs (e.g., 60%, 70%, 80%, 90% or 100%). In certain exemplary embodiments, the second population of TILs comprises an increased subpopulation of CD8+ T cells relative to a population of TILs produced by a pre-REP expansion process in which the first population of TILs is cultured in the pre-REP phase without addition of the population of modified cells of leukemic origin. In certain exemplary embodiments, the subpopulation of CD8+ T cells constitutes at least 30% (e.g., 30%, 40%, 50%, 60%, 70%, 80%) of the total second population of TILs after the pre-REP expansion is performed for about 14 days.

In certain exemplary embodiments, the second population of TILs comprises an increased ratio of the subpopulation of CD8+ T cells to the subpopulation of CD4+ T cells relative to a population of TILs produced by a pre-REP expansion process in which the first population of TILs is cultured without addition of the population of modified cells of leukemic origin.

In certain exemplary embodiments, the second population of TILs exhibits a decreased frequency of CD4+ and CD8+ T cells expressing the exhaustion markers CD272 and PD-1 determined by flow cytometry relative to a population of TILs produced by a pre-REP phase expansion in which the first population of TILs is cultured without addition of the population of modified cells of leukemic origin.

In various embodiments, the population of modified cells of leukemic origin comprises at least one tumor antigen selected from the group consisting of WT-1, RHAMM, PRAME, MUC-1, p53, and Survivin. In certain embodiments, the population of modified cells of leukemic origin is CD34-positive, CD1a-positive, CD83-positive, and CD14-negative. In certain embodiments, the population of modified cells of leukemic origin comprises a co-stimulatory molecule. In certain embodiments, the population of modified cells of leukemic origin is CD40-positive, CD70-positive, CD80-positive, and CD86-positive. In one embodiment, the population of modified cells of leukemic origins is derived from cell line DCOne as deposited under the conditions of the Budapest treaty with the DSMZ under accession number DSMZ ACC3189 on 15 Nov. 2012.

In certain exemplary embodiments, the irradiated allogeneic PBMCs are obtained from at least three healthy donors.

In certain exemplary embodiments, the number of the irradiated allogeneic PBMCs added into the culture at the start of the REP expansion is about 200-fold of the number of co-cultured TILs from the second population of TILs.

In certain exemplary embodiments, the at least one cytokine present in the culture during the REP phase is selected from IL-2, IL-15, IL-21, and/or IL-7. In certain embodiment, at least one cytokine comprises IL-2, and the concentration of IL-2 in the REP phase is from about 1000 IU/mL to about 6000 IU/mL. In one embodiment, the concentration of IL-2 in the REP phase is about 3000 IU/mL.

In certain exemplary embodiments, the second expansion (REP expansion) is performed for a period of up to two weeks. In one embodiment, the rapid expansion is performed for 14 days.

In certain exemplary embodiments, the method results in a greater expansion of TILs from the first population of TILs to the third population of TILs relative to a process including a relative pre-REP expansion and subsequent REP expansion in which the relative pre-REP expansion is performed without addition of the population of modified cells of leukemic origin. In certain exemplary embodiments, the subpopulation of CD8+ T_{EM} cells constitutes about 80% of the CD 8+ T cells in the third population of TILs after performing the first expansion for about 14 days followed by the second expansion for about 14-days.

In certain exemplary embodiments, the subpopulation of CD8+ T_{EMRA}cells constitutes about 10% of the CD 8+ T cells in the third population of TILs after the first expansion is performed for 14 days and the subsequent second expansion is performed for about 14 days, which is lower than the subpopulation of CD8+ T_{EMRA} in a population of TILs produced by a relative process including a relative first expansion for about 14 days and the second expansion for about 14 days in which the relative first expansion is performed without addition of the population of modified cells of leukemic origin.

In certain exemplary embodiments, the method results in a lower frequency of T cells expressing exhaustion markers CD272 and PD-1 in the third population of TILs relative to a process including a relative first expansion and the second expansion in which the relative first expansion is performed without addition of the population of modified cells of leukemic origin.

In certain exemplary embodiments, the third population of TILs exhibits a lower grade of exhaustion of CD8+ T cells as measured by an increased frequency of CD8+ T cells producing IFNγ upon activation with PHA, when the pre-REP phase and the REP phase are respectively performed for 14 days for each phase, relative to a population of TILs produced by a process including a pre-REP expansion and a subsequent REP expansion in which the pre-REP expansion is performed without addition of the population of modified cells of leukemic origin.

In certain exemplary embodiments, the method described above further comprises a step of harvesting and sterilizing the third population of TILs to produce a therapeutic population of TILs suitable for treating a cancer. In certain embodiments, the therapeutic population of TILs comprises sufficient cell number of TILs for effectively treating the cancer. In one embodiment, the cell number of TILs sufficient for an effective dose is from about 10 billion to about 250 billion. In one embodiment, cell number of TILs sufficient for an effective dose is from about 10 million to about 150 billion.

In another aspect, disclosed herein is a method for expansion of tumor infiltrating lymphocytes (TILs) and production of a therapeutic population of TILs for treating cancer, where the method comprises (a) obtaining a tumor sample comprising a first population of TILs from a donor subject having cancer; (b) performing a first expansion of TILs by co-culturing the first population of TILs and a population of modified cells of leukemic origin during a pre-REP phase in a culture comprising interleukin-2 (IL-2) for a first period of time ranging from about 7 days to about 21 days, thereby producing a second population of TILs, wherein the population of modified cells of leukemic origin comprises a mature dendritic cell phenotype and is non-proliferating, and wherein the second population of TILs is greater in number than the first population of TILs; (c) performing a second expansion by culturing the second population of TILs in a REP phase in a culture comprising an anti-CD3 antibody, at least one cytokine, and irradiated allogeneic peripheral blood mononuclear cells (PBMCs) without addition of the population of modified cells of leukemic origin for a second period of up to 2 weeks, thereby producing a third population of TILs, wherein the third population of TILs is greater in number than the second population of TILs; and (d) harvesting and sterilizing the third population of TILs to produce a therapeutic population of TILs suitable for treating a cancer in a receiver subject in need thereof. In certain exemplary embodiments, the donor subject is the receiver subject, and wherein the therapeutic population of TILs is for autologous treatment.

In yet another aspect, disclosed herein is a method for ex vivo stimulation and expansion of tumor infiltrating lymphocytes (TILs), comprising: (a) performing a pre-rapid expansion of TILs by co-culturing a first population of TILs from a tumor sample and a population of modified cells of leukemic origin in a pre-rapid expansion protocol (pre-REP) culture comprising interleukin-2 (IL-2), thereby producing a second population of TILs, wherein the population of modified cells of leukemic origin comprises a mature dendritic cell phenotype and is non-proliferating, and wherein the second population of TILs is greater in number than the first population of TILs. In certain embodiments, the pre-rapid expansion is performed for a period of about 7 days to about 21 days, optionally about 14 days or 21 days. In certain exemplary embodiments, the ratio of the number of the modified cells of leukemic origin added in the first phase expansion culture to the number of TILs in the first population is from about 1:10 to about 1:1.51. In certain embodiments, IL-2 is present in the pre-REP culture at a concentration ranging from about 1000 IU/mL to about 6000 IU/mL. In yet certain embodiments, the first population of TILs is a population of unselected TILs comprised in the tumor sample, and wherein the tumor sample is co-cultured with the population of modified cells of leukemic origin in the pre-REP culture. In one embodiment, TILs are expanded more than 5-fold at the completion of the pre-rapid expansion. In various embodiments, the tumor sample may be from melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma. In certain embodiment. In one embodiment, the second population of TILs is greater in number than a population of TILs produced by culturing the first population of TILs in a pre-REP phase without addition of the population of modified cells of leukemic origin. In yet another embodiment, the second population of TILs comprises an increased subpopulation of CD8+ T cells relative to a population of TILs produced by a pre-REP expansion process in which the first population of TILs is cultured in the pre-REP phase without addition of the population of modified cells of leukemic origin. In still another embodiment, the second population of TILs comprises an increased ratio of the subpopulation of CD8+ T cells to the subpopulation of CD4+ T cells relative to a population of TILs produced by a pre-REP expansion process in which the first population of TILs is cultured without addition of the population of modified cells of leukemic origin. In various embodiment, the population of modified cells of leukemic origin comprises at least one tumor antigen selected from the group consisting of WT-1, RHAMM, PRAME, MUC-1, p53, and Survivin; is CD34-positive, CD1a-positive, CD83-positive, and CD14-negative; and/or is CD40-positive, CD70-positive, CD80-positive, and CD86-positive. In one embodiment, the population of modified cells of leukemic origins is derived from cell line DCOne as deposited under the conditions of the Budapest treaty with the DSMZ under accession number DSMZ ACC 3189 on 15 Nov. 2012.

In certain embodiments, the above method further comprises (b) performing a rapid expansion of TILs by culturing the second population of TILs in a rapid expansion protocol (REP) culture comprising at least one cytokine, without addition of any allogeneic peripheral blood mononuclear cells (PBMCs), thereby producing a third population of TILs, wherein the third population of TILs is greater in number than the second population of TILs. In yet other certain embodiments, the above method further comprises performing a subsequent rapid expansion of TILs by culturing the second population of TILs in a rapid expansion protocol (REP) culture comprising at least one cytokine, without addition of any anti-CD3 antibody, thereby producing a third population of TILs, wherein the third population of TILs is greater in number than the second population of TILs.

In certain embodiment, the at least one cytokine present in the culture during the REP phase is selected from IL-2, IL-15, IL-21, and/or IL-7. In one embodiment, at least one cytokine comprises IL-2, and wherein the concentration of IL-2 in the REP phase is from about 1000 IU/mL to about 6000 IU/mL, optionally wherein the concentration of IL-2 in the REP phase is about 3000 IU/mL. In certain exemplary, the rapid expansion is performed for a period of up to two weeks, optionally wherein the rapid expansion is performed for 14 days.

In certain embodiments, the method described above further comprises a step of harvesting and sterilizing the second population of TILs produced from the pre-REP expansion or the third population of TILs produced from the REP expansion to produce a therapeutic population of TILs suitable for treating a cancer in a receiver subject in need thereof.

In another aspect, disclosed herein is a method for treating a subject having cancer, comprising administering an effective dose of the therapeutic population of TILs produced by the methods disclosed herein for expanding TILs from tumor samples. In certain exemplary embodiments, the effective dose of the therapeutic population of TILs comprises from about 10 billion to about 250 billion TILs. In certain exemplary embodiments, the effective dose comprise from about 10 billion to about 150 billion TILs. In certain exemplary embodiments, the treatment method further comprises administering to the subject an effective amount of an immunomodulatory agent prior to the administration of the therapeutic population of TILs. In certain embodiments, the immunomodulatory agent comprises a cytokine. In certain embodiments, the cytokine is chosen from IL-2, IL-7, IL-15, IL-21, and variants or combinations thereof.

### BRIEF DESCRIPTION OF THE DRAWINGS

The accompanying drawings, which are incorporated herein and constitute part of this specification, illustrate exemplary embodiments of the disclosure, and, together with the general description given above and the detailed description given below, serve to explain the features of the disclosure.

FIG. 1A depicts a set of light microscope cell culture representative images illustrating the pre-REP cultures of ovarian cancer TILs from four patients in the presence of IL-2, with or without the addition of DCOne mature dendritic cells (mDCs), on day 7 from the initiation of the pre-REP phase.

FIGS. 1B and 1C graphically depict the expansion of TILs after the tumor dissociates from four ovarian cancer patients were cultured using the standard pre-REP protocol in the presence of IL-2 (6000 IU/mL), with or without DCOne mDCs, for 14 days (FIG. 1B) and for 21 days (FIG. 1C).

FIG. 1D depicts the expansion of TILs after the tumor dissociates from five endometrial cancer patients were cultured for 14 days using the standard pre-REP protocol in the presence of IL-2 (6000 IU/mL), with or without DCOne mDCs.

FIGS. 1E and 1F graphically depicts the cell viabilities of TILs from ovarian cancer patients as measured using a nucleocounter on day 14 (FIG. 1E) and on day 21 (FIG. 1F) of the pre-REP culture.

FIG. 2A depicts representative flow cytometric plots of CD4+ T cells and CD8+ T cells in pre-REP cultures of TILs from two ovarian cancer donors, with or without stimulation with DCOne mDCs.

FIGS. 2B and 2C graphically depict the immune cell composition on day 14 (FIG. 2B) and on day 21 (FIG. 2C) of pre-REP culture of TILs from ovarian cancer donors, comparing with and without the addition of DCOne mDCs.

FIGS. 2D and 2E graphically depict the cell ratio of CD8+ T cells to CD4+ T cells (CD8:CD4) in the pre-REP cultures on day 14 (FIG. 2D) and on day 21 (FIG. 2E) from the initiation of the pre-REP phase, comparing with and without the addition of DCOne mDCs.

FIGS. 3A and 3B depict the expression of activation and checkpoint molecules on CD3 T cells in day 14 and in day 21 pre-REP cultures, comparing the cultures with or without the addition of DCOne mDCs.

FIGS. 3C and 3D depicts the frequencies of CD4 and CD8 naive (T_{N}), central memory (T_{CM}), effector memory (T_{EM}), and terminally differentiated effector memory (T_{EMRA}) T cells in the cultures with or without DCOne mDCs on day 14 (FIG. 3C) and on day 21 (FIG. 3D) of the pre-REP phase.

FIGS. 4A and 4B depict the total expansion of TILs from day 14 pre-REP (FIG. 4A) and from day 21 (FIG. 4B) pre-REP cultures, with or without the addition of DCOne mDCs, subsequently expanded for 14 days using the standard REP-protocol including irradiated PBMC from 3 different healthy donor in the presence of IL-2 (3000 IU/mL) and anti-CD3 antibody (OKT-3 clone), according to an experimental example disclosed herein.

FIG. 4C depicts the TIL expansion fold obtained in the REP phase, where TILs expanded for 14 days in pre-REP cultures, with or without addition of DCOne mDCs, were subsequently expanded for 14 days using the standard REP-protocol including irradiated PBMC from 3 different healthy donor in the presence of IL-2 (3000 IU/mL) and anti-CD3antibody (OKT-3 clone), according to an experimental example disclosed herein.

FIGS. 4D and 4E depict the frequencies of CD4 and CD8 naive (T_{N}), central memory T_{CM}, effector memory (T_{EM}) and terminally differentiated effector memory (T_{EMRA}) T cells in CD4+ and CD8+ T cells in the final REP cultures after TILs from tumor samples were expanded in the pre-REP phase with or without addition of DCOne mDCs for 14 days (FIGS. 4D) and 21 days (FIG. 4E) and subsequently expanded in the REP phase for 14 days.

FIGS. 4F and 4G depict the expression of activation and inhibition molecules on CD3 T cells in the final REP TIL cultures after TILs from tumor samples were expanded in the pre-REP phase with or without addition of DCOne mDCs for 14 days (FIG. 4F) and for 21 days (FIG. 4G), and subsequently expanded in the standard REP phase without addition of DCOne mDCs for 14 days.

FIGS. 5A-5D depict the frequencies of CD107a expressing CD4+ T cells and CD8+ T cells in the final TIL cultures, where the TILs were expanded in the pre-REP phase with or without the addition of DCOne mDCs for 14 days (FIG. 5A and FIG. 5B) and for 21 days (FIG. 5C and FIG. 5D) and subsequently expanded in the standard REP phase without the addition of DCOne mDCs for 14 days.

FIGS. 5E-5H depict the frequencies of CD4+ T cells and CD8+ T cells producing INFγ upon PHA induction in the final TIL cultures, wherein the TILs were expanded in the pre-REP phase with or without the addition of DCOne mDCs for 14 days (FIG. 5E and FIG. 5F) and for 21 days (FIG. 5G and FIG. 5H) and subsequently expanded in the standard REP phase without the addition of DCOne mDCs for 14 days.

FIG. 6 depicts results of flow cytometry experiments examining INFγ (y-axis) and CD107 (x-axis) expression levels in CD3⁺ Tcells upon co-culture with autologous tumor (left and center scatterplots) or no autologous tumor (control, right scatter plot) in the final TIL cultures, wherein the TILs were expanded in the pre-REP phase with (left scatterplot, right scatterplot) or without (center scatterplot) addition of DCOne mDCs for 14 days and subsequently expanded in the standard REP phase without addition of DCOne mDCs for 14 days.

### DETAILED DESCRIPTION

The present disclosure relates to methods for ex vivo stimulating and expanding tumor infiltrating lymphocytes (TILs) from solid tumors. The methods disclosed herein generally involve utilizing a modified cell of leukemic origin in a pre-rapid expansion phase to enhance ex vivo expansion of TILs. The present disclosure also relates to pharmaceutical compositions comprising the expanded TILs for treating cancer, as well as methods of using the expanded TILs for treating cancer.

It is to be understood that the methods described herein are not limited to particular methods and experimental conditions disclosed herein as such methods and conditions may vary. It is also to be understood that the terminology used herein is for the purpose of describing particular embodiments only and is not intended to be limiting. The methods described herein use conventional molecular and cellular biological and immunological techniques that are well within the skill of the ordinary artisan. Such techniques are well known to the skilled artisan and are explained in the scientific literature.

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

For the disclosure to be more readily understood, select terms are defined below.

The articles “a” and “an” are used herein to refer to one or to more than one (i.e., to at least one) of the grammatical object of the article. By way of example, “an element” means one element or more than one element.

The use of “or” herein is inclusive.

As used herein, the term “about” or “approximately” will be understood by persons of ordinary skill in the art and will vary to some extent on the context in which it is used. When referring to a measurable value such as an amount, a temporal duration, and the like, the term “about” or “approximately” is meant to encompass variations of ±20%, ±15%, or ±10%, including ±5%, ±1%, and ±0.1% from the specified value, as such variations are appropriate to perform the disclosed methods.

“Optional” or “optionally” means that the subsequently described event or circumstance may or may not occur, and that the description includes instances where said event or circumstance occurs and instances where it does not.

As used herein, the term “administering” refers to delivering or applying a composition, an active agent, or a cell, according to the present disclosure, to a subject by any suitable route for delivery of the composition, active agent, or cells to the subject. Examples of routes of administration include, but are not limited to, subcutaneous, intravenous, e.g., intravenous injection and intravenous infusion, e.g., via central venous access, intramuscular, oral, nasal, and pulmonary administration.

“Activation,” as used herein referring to T cells, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “anti-CD3 antibody”, as used herein, refers to an antibody or variant thereof, e.g., a monoclonal antibody, including human, humanized, chimeric or murine antibodies which are directed against the CD3 receptor in the T cell antigen receptor of mature T cells. An anti-CD3 antibody can be OKT-3, also known as muromonab. Anti-CD3 antibodies can also include the UHCT1 clone, also known as T3 and CD3R. Other anti-CD3 antibodies include, for example, otelixizumab, teplizumab, and visilizumab.

As used herein, a “non-tumor antigen” refers to an antigen that is not derived from a tumor. For example, in certain embodiments, a non-tumor antigen may be a foreign antigen. As used herein, a “tumor-independent antigen” refers to an antigen that is not derived from a tumor that a subject is currently suffering from. For example, in certain embodiments, a tumor-independent antigen may be a foreign antigen. A tumor-independent antigen may be human or non-human.

As used herein, the term “allogenic” refers to a material (e.g., tissues or cells) obtained or derived from an individual (donor) who is different from the individual to whom the material is later introduced or delivered (recipient).

As used herein, the term “autologous” refers to a material (e.g., TILs) obtained or derived from the same individual (subject) to whom the material is later re-introduced or delivered back.

A “co-stimulatory ligand” refers to a molecule on an antigen presenting cell that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like.

A “co-stimulatory signal,” as used herein, refers to a signal, which in combination with a primary signal, such as TCR/CD3 ligation, leads to T cell proliferation and/or upregulation or downregulation of key molecules. In certain exemplary embodiments, the co-stimulatory signal is CD70.

As used herein, the term “cryopreserved TILs” refers to TILs, either primary TILs from a patient's tumor tissue that have not been expanded or TILs expanded ex vivo in a lab (either from a pre-REP expansion or a REP expansion), selected TILs or unselected TILs (bulk, or young TILs), that are frozen and stored at a temperature of about −80° C. or lower. “Cryopreserved TILs” are distinguishable from frozen tissue samples that may be used as a source of primary TILs.

As used herein, a “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “dosage” refers to the administering of a specific amount, number, and frequency of doses over a specified period of time. Dosage implies duration. A “dosage regimen” is a treatment plan for administering a drug over a period of time.

As used herein, the terms “effective amount” and “therapeutically effective amount” are interchangeable and refer to an amount of therapeutic cells, such as TILs described herein, or compositions comprising the therapeutic cells, that is of sufficient quantity to provide a particular biological result or provides a therapeutic or prophylactic benefit. Such results or benefits may include, but are not limited to, a detectable level of immune suppression or tolerance compared to the immune response detected in the absence of the composition of the disclosure. The immune response can be readily assessed by a plethora of art-recognized methods. The results or benefits may also include ameliorating one or more causes or symptoms of a disease or disorder. Such amelioration only requires a reduction or alteration, not necessarily elimination. A skilled artisan would understand that the amount may vary and can be determined based on a number of factors such as the disease or condition being treated, the age and health and physical condition of the subject being treated, the severity of the disease, the manner of administration, and the like. In certain circumstance, the optimal amount of the compositions of the present disclosure to be administered can be determined by a physician with consideration of individual differences in age, weight, tumor size, extent of infection or metastasis, and condition of the patient (subject).

As used herein “endogenous” refers to any material from or produced inside an organism, cell, tissue or system.

As used herein, the term “exogenous” refers to any material introduced from or produced outside an organism, cell, tissue or system.

As used herein, the term “expand” refers to increasing in number of cells, as in an increase in the number of TILs, including T cells. In one embodiment, the cells that are expanded ex vivo in a culture increase in number, relative to the number originally present in the culture.

The term “ex vivo” refers to an event which involves treating or performing a procedure on a cell, tissue and/or organ which has been removed from a subject's body. For instance, cells may be removed from a living organism(e.g., from a non-human animal or a human) and propagated outside the organism (e.g., in a culture dish, test tube, or bioreactor). The cell, tissue and/or organ may be returned to the subject's body in a method of surgery or treatment. The term “in vitro” refers to an event that takes places outside of a subject's body. In vitro assays may encompass cell-based assays in which cells alive or dead are employed and may also encompass a cell-free assay in which no intact cells are employed.

The term “in vivo” refers to an event that takes place in a subject's body.

As used herein, “feeder cells” refer to cells that are unable to divide or proliferate but can help the growth of other cells by releasing certain growth factors and/or extracellular secretions. In certain situations, feeder cells may be irradiated peripheral blood mononuclear cells from healthy donors, which are used to support the expansion and activation of TILs during the culturing process and providing necessary costimulatory signals for optimal T-cell proliferation. In certain situations, feeder cells may be artificial antigen-presenting cells (aAPCs).

As used herein, the term “immune response” includes T cell mediated and/or B cell mediated immune responses. Exemplary immune functions of T cells include, e.g., cytokine production and induction of cytotoxicity in other cells. B cell functions include antibody production. In addition, the term includes immune responses that are indirectly affected by T cell activation, e.g., antibody production and activation of cytokine responsive cells, e.g., macrophages. Immune cells involved in the immune response include lymphocytes, such as B cells and T cells (CD4⁺ and CD8⁺ cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes. In certain embodiments, the term refers to a T cell mediated immune response. The immune response may in some embodiments be a T cell-dependent immune response. As used herein, the term “T cell dependent immune response” refers to an immune response wherein either T cells, B cells or both T cell and B cell populations are activated, and wherein T cells further assist T and B cells and other immune cells in executing their function. A skilled person in the art understands that the phrase “immune response against a tumor” also includes immune responses against a non-human antigenic polypeptide that is introduced into the tumor micro-environment by intratumoral administration, such as intratumoral administration of (i) dendritic cells, including autologous or allogeneic dendritic cells, loaded with said polypeptide or (ii) viruses comprising a nucleic acid encoding said polypeptide.

The terms “fragmenting,” “fragment,” and “fragmented,” as used herein for describing a process for disrupting a tumor, refer to mechanically cutting, slicing, crushing, dividing, and morcellating the tumor tissue into small pieces.

As used herein, the term “immunosuppressive” refers to reducing overall immune response. “Immunosuppressive cells” are cells that facilitate tumor immune escape by inhibiting antitumor immune responses and promote tumor metastasis by inducing immunosuppression, promoting tumor cell invasion and intravasation, establishing a pre-metastatic niche, facilitating epithelial-mesenchymal transition, and inducing angiogenesis at primary tumor or metastatic sites.

As used herein, the terms “level” and “levels” can be used interchangeably with the terms “concentration” and “concentrations.”

As used herein, the term “modulating” refers to mediating a detectable increase or decrease in the level of a response in a subject compared with the level of a response in the subject in the absence of a treatment or compound, and/or compared with the level of a response in an otherwise identical but untreated subject. This term encompasses perturbing and/or affecting a native signal or response thereby mediating a beneficial therapeutic response in a subject, e.g., a human.

By the term “modified” as used herein, is meant a changed state or structure of a molecule or cell of the disclosure. Molecules may be modified in many ways, including chemically, structurally, and functionally. Cells may be modified through the introduction of nucleic acids.

As used herein, the term “parenteral” administration refers to the administration of a drug such as an immunogenic composition in a manner other than through the digestive tract. Parenteral administration includes, e.g., subcutaneous (s.c.), intravenous (i.v.), intramuscular (i.m.), intradermal, intraperitoneal, or intrasternal injection, or infusion techniques.

“Peripheral blood mononuclear cells” and “PBMCs” are a heterogeneous population of blood cells with a single round nucleus, which include macrophages, dendritic cells, monocytes, and lymphocytes (T cells, B cells, NK cells, etc.).

As used herein, the term “pre-rapid expansion protocol (pre-REP)” phase refers to the first phase (or stage) of a two-phase (or two-stage) process for expanding tumor-infiltrating lymphocytes (TILs) ex vivo. A pre-REP phase is also called the “first expansion phase” or “pre-REP stage” herein. A standard pre-REP protocol typically involves an initial expansion of TILs by culturing primary TILs or tumor fragments or digested tumor tissues in the presence of IL-2, without the addition any modified cell of leukemic origin as described herein. In contrast, the pre-REP phase disclosed herein is carried out by co-culturing primary TILs from a tumor with a population of modified cell of leukemic origin in the presence of IL-2, where IL-2 is present in the culture at a high concentration, such as 1000 IU/mL or above. In certain embodiments, medium exchanges with fresh IL-2 may be performed regularly to ensure continued T-cell division and survival during this time. The pre-REP expansion yields a first product (also called “pre-REP” TILs), which can be then further expanded into a final population of TILs using a “rapid expansion protocol” (REP). In certain situations, TILs expanded over the pre-REP phase (pre-REP TILs) can be immediately expanded using the REP protocol in a subsequent expansion phase (i.e., a second expansion phase, or REP phase). Alternatively, the pre-REP TILs can be cryopreserved at the end of the pre-REP stage for a later secondary expansion in REP and patient treatment or can be used immediately.

As used herein, the term “pre-REP TILs” refers to TILs produced at the completion of the pre-REP expansion.

As used herein, the term “rapid expansion protocol (REP)” phase refers to a subsequent or second phase (or second stage) following the pre-REP phase in a two-phase process for expanding tumor-infiltrating lymphocytes (TILs) ex vivo. The REP stage involves quickly activating and expanding TILs. As such, in certain situations, the REP phase may be simply called “a rapid expansion phase.” A standard REP protocol is generally performed by culturing TILs in a culture medium comprising irradiated peripheral blood mononuclear cells or PBMCs (feeder cells), at least one anti-CD3 antibody (e.g., OKT-3), and high dose IL-2, such as a dose of at least 1000 IU/mL IL-2. PBMC feeder cells are generally obtained normal healthy donors (allogeneic feeders), with a preference of more than one or two healthy donors. Typically, TILs are expanded significantly more during the REP phase compared to the pre-REP phase. In certain situations, for convenience, TILs produced at the end of the REP phase can also be called “REP TILs”.

As used herein, a “pharmaceutically acceptable” component refers to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

As used herein, the term “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “stimulation” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

As used herein, the term “stimulatory molecule,” means a molecule on an immune cell, e.g., T cell, that specifically binds with a cognate stimulatory ligand present on an antigen presenting cell.

A “stimulatory ligand,” as used herein, means a ligand that when present on an antigen presenting cell (e.g., an aAPC, a dendritic cell, a B cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject,” as used herein, refers to the recipient of a method as described herein, i.e., a recipient that can mount a cellular immune response, and is a mammal. In certain embodiments, the subject is a human. In certain embodiments, the subject is a domesticated animal, e.g., a horse, a cow, a pig, a sheep, a dog, a cat, etc. The terms “patient” and “subject” may be used interchangeably. In certain embodiments, the subject is a human suffering from a tumor (e.g., a solid tumor). In certain embodiments, the subject is a domesticated animal suffering from a tumor (e.g., a solid tumor).

The term “subject,” as used herein, refers to an individual who has a particular solid tumor or cancer. The subject may be a donor of tumor tissues for expanding TILs and the recipient of the expanded TILs expanded, where the expanded TILs are delivered back, e.g., infused back. In certain circumstances, the recipient can mount a cellular immune response. In certain circumstances, the subject is a mammal. In certain embodiments, the subject is a human. In certain embodiments, the subject can be a non-human animal, e.g., a non-human primate, a horse, a cow, a pig, a sheep, a dog, a cat, etc. The terms “patient” and “subject” may be used interchangeably. In certain embodiments, the subject is a human suffering from a tumor (e.g., a solid tumor). In certain embodiments, the subject is a domesticated animal suffering from a tumor (e.g., a solid tumor).

As used herein, the term “subject in need thereof” refers to a subject in need of a treatment of a disease or condition. In various embodiments, a “subject in need thereof” may have an on-going cancer or condition such as ovarian cancer, endometrial cancer, or other solid tumors. In certain situations, a “subject in need thereof” may have received an initial treatment for a cancer such as surgery, chemotherapy, and/or radiation therapy and is in partial or complete remission before receiving the TILs therapy described herein. In certain situations, “a subject in need thereof” may be a subject having received an initial treatment as described above and is in remission with minimal tumor cells (i.e., the subject has minimal residual disease “MRD” that can potentially lead to a relapse) before receiving the TILs therapy described herein.

The term “therapeutic” as used herein means a treatment and/or prophylaxis. A therapeutic effect is obtained by suppression, remission, or eradication of a disease state.

As used herein, the term ‘treat,” “treatment,” “treating,” or “amelioration” refers to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well.

“Tumor infiltrating lymphocytes” or “TILs” refer to a population of immune cells that has moved from the blood into a tumor. TILs include, but are not limited to, CD8⁺ cytotoxic T cells (lymphocytes), CD4⁺ T cells, natural killer cells, dendritic cells, and M1 macrophages. TILs may be primary TILs or secondary TILs. The term “primary TILs” refers to TILs obtained from patient tumor tissue samples and have not been stimulated, proliferated, expanded, or modified ex vivo. In certain circumstance, TILs may be genetically modified.

The term “tumor,” as used herein, includes reference to cellular material, e.g., a tissue, proliferating at an abnormally high rate. A growth comprising neoplastic cells is a neoplasm, also known as a “tumor,” and generally forms a distinct tissue mass in a body of a subject. A tumor may show partial or total lack of structural organization and functional coordination with the normal tissue. As used herein, a tumor is intended to encompass hematopoietic tumors as well as solid tumors. In certain embodiments, the tumor is a solid tumor. The term “tumor” further includes reference to the tumor micro-environment or tumor site, i.e., the area within the tumor and the area directly outside the tumorous tissue. In certain embodiments, the tumor micro-environment or tumor site includes an area within the boundaries of the tumor tissue. In certain embodiments, the tumor micro-environment or tumor site includes the tumor interstitial compartment of a tumor, which is defined herein as all that is interposed between the plasma membrane of neoplastic cells and the vascular wall of the newly formed neovessels. As used herein, the terms “tumor micro-environment” or “tumor site” refers to a location within a subject in which a tumor resides, including the area immediately surrounding the tumor.

A tumor may be benign (e.g., a benign tumor) or malignant (e.g., a malignant tumor or cancer). Malignant tumors can be broadly classified into three major types: those arising from epithelial structures are called carcinomas, those that originate from connective tissues such as muscle, cartilage, fat or bone are called sarcomas, and those affecting hematopoietic structures (structures pertaining to the formation of blood cells) including components of the immune system, are called leukemias and lymphomas. Other tumors include, but are not limited to, neurofibromatosis.

Solid tumors are abnormal masses of tissue that can be benign or malignant. In certain embodiments, solid tumors are named for the type of cells that form them (such as sarcomas, carcinomas, and lymphomas). Examples of solid tumors, such as sarcomas and carcinomas, include, but are not limited to, liposarcoma, fibrosarcoma, chondrosarcoma, osteosarcoma, myxosarcoma, and other sarcomas, mesothelioma, synovioma, leiomyosarcoma, Ewing's tumor, colon carcinoma, rhabdomyosarcoma, pancreatic cancer, lymphoid malignancy, lung cancers, breast cancer, prostate cancer, ovarian cancer, hepatocellular carcinoma, adenocarcinoma, basal cell carcinoma, sweat gland carcinoma, squamous cell carcinoma, medullary thyroid carcinoma, pheochromocytomas sebaceous gland carcinoma, papillary thyroid carcinoma, papillary adenocarcinomas, papillary carcinoma, medullary carcinoma, bronchogenic carcinoma, hepatoma, renal cell carcinoma, bile duct carcinoma, Wilms' tumor, choriocarcinoma, cervical cancer, seminoma, testicular tumor, bladder carcinoma, melanoma, CNS tumors (e.g., a glioma, e.g., brainstem glioma and mixed gliomas, glioblastoma (e.g., glioblastoma multiforme), germinoma, astrocytoma, craniopharyngioma, medulloblastoma, ependymoma, Schwannoma, CNS lymphoma, acoustic neuroma, pinealoma, hemangioblastoma, meningioma, oligodendroglioma, retinoblastoma, neuroblastoma, and brain metastases), and the like. In certain exemplary embodiments, the tumor is a glioblastoma. In certain exemplary embodiments, the tumor is an ovarian cancer (e.g., an epithelial ovarian cancer, which can be further subtyped into a serous, a clear cell, an endometrioid, a mucinous, or a mixed epithelial ovarian cancer).

Carcinomas that can be amenable to therapy by a method disclosed herein include, but are not limited to, squamous cell carcinoma (various tissues), basal cell carcinoma (a form of skin cancer), esophageal carcinoma, bladder carcinoma, including transitional cell carcinoma (a malignant neoplasm of the bladder), hepatocellular carcinoma, colorectal carcinoma, bronchogenic carcinoma, lung carcinoma, including small cell carcinoma and non-small cell carcinoma of the lung, colon carcinoma, thyroid carcinoma, gastric carcinoma, breast carcinoma, ovarian carcinoma, adrenocortical carcinoma, pancreatic carcinoma, sweat gland carcinoma, prostate carcinoma, papillary carcinoma, adenocarcinoma, sebaceous gland carcinoma, medullary carcinoma, papillary adenocarcinoma, ductal carcinoma in situ or bile duct carcinoma, cystadenocarcinoma, renal cell carcinoma, choriocarcinoma, Wilm's tumor, seminoma, embryonal carcinoma, cervical carcinoma, testicular carcinoma, nasopharyngeal carcinoma, osteogenic carcinoma, epithelial carcinoma, uterine carcinoma, and the like.

Sarcomas that can be amenable to therapy by a method disclosed herein include, but are not limited to, myxosarcoma, chondrosarcoma, chordoma, osteogenic sarcoma, liposarcoma, fibrosarcoma, angiosarcoma, lymphangiosarcoma, endotheliosarcoma, osteosarcoma, mesothelioma, Ewing's sarcoma, leiomyosarcoma, rhabdomyosarcoma, lymphangioendotheliosarcoma, synovioma, and other soft tissue sarcomas.

The term “modified cell of leukemic origin,” as used herein, refers to a cell that can take up an antigen such as an antigenic polypeptide into its cell, and presents the antigen, or an immunogenic part thereof together with an MHC class I complex or MHC class II complex. In certain embodiments, the modified cell of leukemic origin is a cell derived from cell line DCOne as deposited under the conditions of the Budapest treaty with the DSMZ under accession number DSMZ ACC 3189 on 15 Nov. 2012. The process of obtaining mature cells from the deposited DCOne cell line is, for instance, described in EP2931878B1 and U.S. Pat. No. 10,064,923, which are incorporated by reference herein. For example, mature dendritic cells can be obtained by incubating the DCOne precursor cell line under conditions that allow differentiation of the progenitor cells into immature dendritic cells and incubating said immature dendritic cells under conditions that allow maturation of the immature dendritic cells into modified cells having a mature dendritic cell phenotype.

The skilled person is well aware of the conditions that allow the differentiation of DCOne progenitor cells into immature dendritic cells. For that purpose, the cells may be contacted with the appropriate stimulatory molecules. The term “stimulatory molecules” relates to compounds capable of inducing differentiation and/or maturation of the cells. Well known examples of stimulatory molecules that may be used for the differentiation of progenitor cells into immature dendritic cells include, cytokines such as IL-4 (Interleukin 4), IL-6, PGE-2 (Prostaglandin E2), TNFalpha (Tumor Necrosis Factor Alpha) and TGF-beta(transforming growth factor beta). Also known are growth factors such as GM-CSF (Granulocyte-macrophage colony-stimulating factor). Surrogate molecules for cytokines or growth factors which induce a biological effect comparable to that of the above stimulatory molecules may also be employed. Such surrogate molecules include antibodies and other biological molecules such as lipopolysaccharides (LPS) and Polyinosinic-polycytidylic acid (polyIC). In certain exemplar embodiments, the precursor cells with a composition comprising GM-CSF, IL-4, and TNF-α.

The person skilled in the art is equally well aware of methods available in the art for obtaining mature dendritic cells from the immature dendritic cells described above. For example, immature dendritic cells may be matured by contacting the immature dendritic cells with stimulatory molecules, such as TNF-alpha, IL-6, IL-1 beta and/or PGE2, although other methods known in the art to mature immature dendritic cells can likewise be employed. Such treatment will allow for obtaining mature dendritic cells from immature dendritic cells. In certain exemplary embodiment, the immature cells are contacted with a composition comprising TNF-α, PGE2, and IL1-β.

The modified cells of leukemic origin thus obtained are fully functional as dendritic cells as evidenced by the expression of high levels of MHC Class I, MHC Class II and CD83, the latter being a typical marker for mature DCs. Such mature DC have the capacity to prime an immune response. In certain embodiments, the modified cells are CD34-positive, CD1a-positive, CD83-positive, and CD14-negative. In certain embodiments, the modified cell of leukemic origin is off-the-shelf leukemic cell-dendritic cell comprising a mature dendritic cell phenotype (DCOne mDC), which strongly expresses crucial T cell co-stimulatory ligands including CD40, CD70, CD80 and CD86. In certain embodiments, the DCOne mDC cells endogenously express at least one tumor antigen, selected from the group consisting of WT-1, RHAMM and PRAME, p53 and Survivin. In certain embodiments, the mature DCOne derived DCs also express HLA-A2, HLA-A3, and HLA-B44 molecules that allow expression of peptides derived from tumor antigens such as WT-1, to be presented to T cells.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to be inclusive and include all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Modified Cell of Leukemic Origin

Disclosed herein provides a specific strategy for efficiently and reproducibly expanding TILs derived from solid tumor tissues. Specifically, the strategy involves co-culturing TILs with a population of modified cells of leukemic origin during the pre-rapid expansion protocol (pre-REP) phase.

As used herein, the term “modified cell of leukemic origin” refers to a cell capable of taking up an antigen such as an antigenic polypeptide, and capable of presenting the antigen, or an immunogenic part thereof, together with an MHC class I complex or MHC class II complex. A modified cell of leukemic origin provided herein comprises a mature dendritic cell phenotype. The term “dendritic cell,” as used herein, refers to a professional antigen presenting cell (APC) that can take up an antigen such as an antigenic polypeptide into its cell, and presents the antigen, or an immunogenic part thereof together with an MHC class I complex or MHC class II complex. Having a mature dendritic cell phenotype means that the modified cell of leukemic origin is capable of performing similar functions to those of a mature dendritic cell. The term includes both immature dendritic cells (“imDCs”) and mature dendritic cells (“mDCs”), depending on maturity.

In certain embodiments, the modified cell of leukemic origin is derived from leukemia cells from a patient having leukemia. In certain embodiments, the modified cell of leukemic origin is derived from the peripheral blood of a patient having leukemia. In certain embodiments, the modified cell of leukemic origin is derived from the peripheral blood of a patient having acute myeloid leukemia. The skilled artisan will recognize that a modified cell of leukemic origin can be derived from any patient obtained peripheral blood, wherein the patient has any type of leukemia, given that the modified cell of leukemic origin thus derived comprises the characteristics disclosed herein.

In certain embodiments, the modified cell of leukemic origin is CD34-positive, CD1a-positive, and CD83-positive. In certain embodiments, the modified cell of leukemic origin comprises a cell surface marker selected from the group consisting of CD14, DC-SIGN, Langerin, CD40, CD70, CD80, CD83, CD86, and any combination thereof. In certain embodiments, the modified cell of leukemic origin comprises an MHC class I molecule. In certain embodiments, the modified cell of leukemic origin comprises an MHC class II molecule. In certain embodiments, the modified cell of leukemic origin is CD34-positive, CD1a-positive, CD83-positive, and CD14-negative. In certain embodiments, the modified cell of leukemic origin is CD40-positive, CD80-positive, and CD86-positive. In certain embodiments, the modified cell of leukemic origin is CD34-positive, CD1a-positive, CD83-positive, CD40-positive, CD80-positive, CD86-positive, and CD14-negative.

In certain embodiments, the modified cell of leukemic origin comprises a genetic aberration between chromosome 11p15.5 to 11p12. In certain embodiments, the genetic aberration encompasses about 16 Mb of genomic regions (e.g., from about 20.7 Mb to about 36.6 Mb). In certain embodiments, the genetic aberration contains a loss of about 60 known and unknown genes.

In certain embodiments, the modified cell of leukemic origin comprises a co-stimulatory molecule. In certain embodiments, the co-stimulatory molecule includes, without limitation, an MHC class I molecule, B and T lymphocyte attenuator (BTLA), and Toll ligand receptor. Examples of co-stimulatory molecules include CD112, CD155, CD70, CD80, CD86, 4-1BBL (CD137-ligand), OX40L, CD30L, CD40, PD-L1, ICOSL, ICAM-1, lymphocyte function-associated antigen 3 (LFA3 (CD58)), K12/SECTM1, LIGHT, HLA-E, B7-H3 and CD83. In certain embodiments, the co-stimulatory molecule is selected from CD112, CD155, and/or CD58.

In certain embodiments, the modified cell of leukemic origin comprises at least one endogenous antigen. Depending on the leukemic origin of the modified cell, the modified cell of leukemic origin may comprise at least one known endogenous antigen that is specific to the leukemic origin. In certain embodiments, the endogenous antigen is a tumor-associated antigen. In certain embodiments, an endogenous tumor-associated antigen may be selected from the group consisting of WT-1, RHAMM, PRAME, p53, Survivin, and MUC-1.

In certain embodiments, the modified cell of leukemic origin is not loaded with any exogenous antigen or peptide fragments thereof.

In certain embodiments, the modified cell of leukemic origin comprises an exogenous antigen or peptide fragments thereof. Such an exogenous antigen may be provided to the modified cell of leukemic origin via various antigen loading strategies. For example, strategies for loading a modified cell of leukemic origin may include, without limitation, the use of synthetic long peptides, mRNA loading, peptide-pulsing, protein-loading, tumor lysate-loading, coculturing with a tumor cell, RNA/DNA transfection or viral transduction. Other strategies for loading a modified cell of leukemic origin are known to those of skill in the art and may be used to load a modified cell of leukemic origin with an exogenous antigen. In general, the modified cell of leukemic origin will process the exogenous antigen via particular molecules, e.g., via MHC I or MHC II. As such, an exogenous antigen comprised by the modified cell of leukemic origin may be an MHC class I antigen or an MHC class II antigen. In certain embodiments, the exogenous antigen is a tumor-associated antigen. For example, in certain embodiments, the modified cell of leukemic origin is loaded with NY-ESO-1 peptide and/or WT-1 peptide, or a tumor-independent antigen such as CMVpp65, CRM197, or variants thereof. In certain embodiments, the exogenous antigen is associated with a disease or disorder, e.g., a non-cancer-associated disease or disorder. It will be appreciated by those of ordinary skill in the art that any tumor-associated antigen or antigen associated with a disease or disorder can be provided to the modified cell of leukemic origin described herein. As such, in certain embodiments, a modified cell of leukemic origin comprises any tumor-associated antigen or antigen associated with a disease or disorder contemplated by those skilled in the art.

In certain embodiments, loading a modified cell of leukemic origin with an exogenous antigen or peptide fragments thereof, includes use of a photochemical processes (e.g., photochemical internalization). In certain embodiments, loading a modified cell of leukemic origin with an exogenous antigen or peptide fragments thereof is achieved with the use of photochemical internalization. In certain embodiments, photochemical internalization may be used to enhance the delivery of an antigen or peptide fragments thereof (e.g., an antigenic polypeptide (e.g., a non-tumor antigen), or a nucleic acid encoding the antigenic polypeptide) into the modified cell of leukemic origin. Photochemical internalization refers to a delivery method which involves the use of light and a photosensitizing agent for introducing otherwise membrane-impermeable molecules into the cytosol of a target cell, but which does not necessarily result in destruction or death of the target cell. In this method, the molecule to be internalized or transferred is applied to the cells in combination with a photosensitizing agent. Exposure of the cells to light of a suitable wavelength activates the photosensitizing agent which in turn leads to disruption of the intracellular compartment membranes and the subsequent release of the molecule into the cytosol. In photochemical internalization, the interaction between the photosensitizing agent and light is used to affect the cell such that intracellular uptake of the molecule is improved. Photochemical internalization as well as various photosensitizing agents are described in PCT Publication Nos. WO 1996/007432, WO 2000/054708, WO 2001/018636, WO 2002/044396, WO 2002/044395, and WO 2003/020309, U.S. Pat. Nos. 6,680,301, and 5,876,989, the disclosures of which are incorporated by reference herein in their entireties. In certain embodiments, photochemical internalization is used to deliver an antigen into the cytosol of a tumor cell. In certain embodiments, photochemical internalization is used to enhance the delivery of an antigen into the cytosol of a tumor cell.

Loading the modified cell of leukemic origin with an exogenous antigen or peptide fragments thereof may be performed at any time. The skilled person will be able to determine and carry out the specific timing of loading of the modified cell of leukemic origin to best suit their needs. For example, in certain embodiments, the modified cell of leukemic origin is loaded with an exogenous antigen or peptide fragments thereof prior to its exhibiting a mature dendritic cell phenotype. In certain embodiments, the modified cell of leukemic origin is loaded with the exogenous antigen or peptide fragments thereof during transition of the modified cell of leukemic origin to a mature dendritic cell phenotype. In certain embodiments, the modified cell of leukemic origin is loaded with the exogenous antigen or peptide fragments thereof after the modified cell of leukemic origin exhibits a mature dendritic cell phenotype.

In certain embodiments, the modified cell of leukemic origin is a cell of cell line DCOne as described in PCT Publication Nos. WO 2014/006058 and WO 2014/090795, the disclosures of which are incorporated by reference herein in their entireties. In certain embodiments, modified cell of leukemic origin is a cell of cell line DCOne and comprises a mature dendritic cell phenotype that is CD34-positive, CD1a-positive, and CD83-positive. In certain embodiments, the modified cell of leukemic origin is a cell of cell line DCOne and is CD34-positive, CD1a-positive, and CD83-positive. In certain embodiments, modified cell of leukemic origin is a cell of cell line DCOne and comprises a cell surface marker selected from the group consisting of CD14, DC-SIGN, Langerin, CD80, CD86, CD40, CD70, and any combination thereof. In certain embodiments, modified cell of leukemic origin is a cell of cell line DCOne and comprises MHC class I. In certain embodiments, modified cell of leukemic origin is a cell of cell line DCOne and comprises MHC class II. In certain embodiments, the modified cell of leukemic origin is a cell of cell line DCOne and is CD34-positive, CD1a-positive, CD83-positive, and CD14-negative. In certain embodiments, the modified cell of leukemic origin is a cell of cell line DCOne and is CD40-positive, CD80-positive, and CD86-positive. In certain embodiments, the modified cell of leukemic origin is a cell of cell line DCOne and is CD34-positive, CD1a-positive, CD83-positive, CD40-positive, CD80-positive, CD86-positive, and CD14-negative. In certain embodiments, modified cell of leukemic origin is a cell of cell line DCOne and comprises a genetic aberration between chromosome 11p15.5 to 11p12. In certain embodiments, modified cell of leukemic origin is a cell of cell line DCOne and comprises a genetic aberration that encompasses about 16 Mb of genomic regions (e.g., from about 20.7 Mb to about 36.6 Mb). In certain embodiments, modified cell of leukemic origin is a cell of cell line DCOne and comprises a genetic aberration that contains a loss of about 60 known and unknown genes.

In certain embodiments, the modified cell of leukemic origin is a plasmacytoid dendritic human cell lines (pDC) as described in U.S. Pat. Nos. 7,341,870 and 9,783,782, which are specifically incorporated herein by reference. In particular embodiments, the plasmacytoid dendritic human cell line is the cell line designated GEN2.2 which is deposited in CNCM (Collection Nationale de Cultures de Microorganismes [National Collection of Cultures of Microorganisms], Pasteur Institute, 25 rue du Docteur Roux, F-75015 Paris) under number CNCM 1-2938. In other embodiments, the plasmacytoid dendritic human cell line is the cell line designated GEN 3, which with deposited with the CNCM under the CNCM number 1-3110.

As provided herein, certain methods are directed to the use of a modified cell of leukemic origin, wherein the modified cell is non-proliferating. In certain embodiments, the modified cell of leukemic origin has been irradiated. In certain embodiments, the modified cell of leukemic origin has been irradiated prior to its use in a method disclosed herein. Irradiation can, for example, be achieved by gamma irradiation at 30-150 Gy, e.g., 100 Gy, for a period of 1 to 3 hours, using a standard irradiation device (Gammacell or equivalent). Irradiation ensures that any remaining progenitor cell in a composition comprising the modified cell of leukemic origin, e.g., a CD34 positive cell, cannot continue dividing. The cells may, for example, be irradiated prior to injection into patients, when used as a vaccine, or immediately after cultivating is stopped. In certain embodiments, the cells are irradiated to inhibit their capacity to proliferate and/or expand, while maintaining their immune stimulatory capacity.

### Sources of TILs for Expansion

In various embodiments according to the present disclosures, TILs are expanded from tumor tissues. Tumor tissues can be obtained from a subject having a cancer by surgical resection, needle biopsy, core biopsy, small biopsy, or any other suitable techniques. In certain situations, the tumor tissues may include a tissue approximately surrounding, or immediately bordering, a tumor.

Typically, after a tumor is excised by surgery from a subject, the tumor is mechanically cut into small pieces. The tumor pieces can be further digested by adding enzymes, such as collagenases, DNAse, hyaluronidase, etc. In certain embodiments, a tumor tissue is separated into single cells using mechanical, enzymatic, and/or chemical dissociation techniques, and a single-cell suspension is prepared.

In certain embodiments, a tumor tissue excised from a subject can be cryopreserved before subjecting to mechanical fragmentation and enzymatical digestion. In certain embodiments, a tumor tissue freshly excised from a subject can be immediately dissociated to prepare a tumor sample for TIL expansion. In one embodiment, dissociated tumor sample can be immediately used for expansion of TILs. Alternatively, tumor dissociates can be cryopreserved for a period of time and be used for a later TIL expansion.

In certain embodiments, an expansion process is carried out with primary TILs that are selected from a tumor tissue. As used herein. “primary TILs” are TILs from a tumor tissue that have not been stimulated, proliferated, expanded, or modified ex vivo. In various embodiments, primary TILs may be selected and isolated from tumor tissues by Ficoll density gradient centrifugation or a specific cell sorting method, such as magnetic-or fluorescence-activated cell sorting (MACS/FACS) (Kazemi et al, Frontiers in Immunology, 2022, 13:1018962), or any other suitable methods known in the art.

In certain embodiments, primary TILs are not selected and isolated from a tumor tissue during expansion. In such situations, a tumor tissue processed by fragmentation, enzymatic, and/or chemical dissociation is directly used for expansion of TILs.

In various embodiments, TILs may be expanded from various types of tumor tissues, such as melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma, as well as any solid tumors defined above. In one embodiment, the tumor is ovarian cancer. In another embodiment, the tumor is endometrial cancer.

In one embodiment, the tumor sample is from a human patient having a particular cancer. In one embodiment, the tumor sample is from a subject who is a non-human animal having a particular cancer.

### Expansion of TILS

### Use of Modified Cells of Leukemic Origin in Pre-REP Phase

Disclosed herein provides a method for ex vivo stimulation and expansion of TILs, where the method includes a new pre-rapid expansion protocol (pre-REP) for stimulation and expansion of tumor infiltrating lymphocytes (TILs) from a tumor sample. The new pre-REP protocol generally involves adding a population of modified cells of leukemic origin in the standard pre-REP culture, and co-culturing TILs form the tumor sample with the modified cells of leukemic origin in the presence of IL-2. The population of modified cells of leukemic origin comprises a mature dendritic cell phenotype and is non-proliferating (e.g., via irradiation).

The new pre-REP protocol can be used in an expansion process comprising at least a pre-rapid expansion phase for expanding TILs from a tumor sample. In certain embodiments, the new pre-REP protocol is used in an expansion process comprising a pre-rapid expansion phase and a subsequent rapid expansion phase. In certain embodiments, the pre-rapid expansion using the new pre-REP protocol disclosed herein is followed with a standard rapid expansion protocol (REP) phase. In the standard REP protocol, the modified cells of leukemic origin are not added in the REP culture, and instead, irradiated allogeneic peripheral blood mononuclear cells (PBMCs) are used as feeder cells in the REP culture. In certain other embodiments, the standard REP protocol may be replaced with a modified REP protocol.

In certain embodiments, disclosed are methods for ex vivo stimulation and expansion of TILs comprising (a) performing a pre-rapid expansion (pre-REP expansion) of TILs by co-culturing a first population of TILs from a tumor sample and a population of modified cells of leukemic origin in a pre-rapid expansion protocol (pre-REP) culture comprising interleukin-2 (IL-2), thereby producing a second population of TILs (pre-REP TILs), wherein the population of modified cells of leukemic origin comprises a mature dendritic cell phenotype and is non-proliferating (e.g., via irradiation), and wherein the second population of TILs is greater in number than the first population of TILs.

In certain embodiments, the methods further comprises (b) performing a subsequent rapid expansion (REP expansion) of TILs by culturing the second population of TILs, without addition of the population of modified cells of leukemic origin, in a rapid expansion protocol (REP) culture comprising an anti-CD3 antibody, at least one cytokine, and irradiated allogeneic peripheral blood mononuclear cells (PBMCs), thereby producing a third population of TILs (REP TILs), wherein the third population of TILs is greater in number than the second population of TILs.

In certain embodiment, the methods disclosed herein further comprise a step of contacting the first population of TILs and the population of modified cells of leukemic origin prior to performing the pre-REP expression.

In certain embodiments, the first population of TILs are primary TILs not selected or isolated from a dissociated tumor sample as described herein. In this situation, step (a) of the method is carried out by co-culturing a dissociated tumor sample comprising the first population of TILs with the population of modified cells of leukemic origin in the pre-REP culture comprising IL-2. In certain embodiments, the first population of TILs are primary TILs selected from the tumor sample. In one embodiment, dead cells are removed from the first population of the TILs prior to performing the pre-REP expansion.

In certain embodiment, the methods disclosed herein further comprise a step of obtaining tumor tissue removed from a subject that has a particular solid tumor, and enzymatically digesting the tumor tissues to prepare a digested tumor sample as a starting cell population for the pre-rapid expansion (the first expansion).

In certain embodiments, the pre-REP expansion is performed for a period of about 7 days to about 21 days. In one embodiment, the pre-REP expansion is performed for about 14 days or about 21 days. In certain embodiments, TILs produced at the completion of the pre-REP phase are further expanded in the REP phase for a period of about two weeks or less.

The modified cells of leukemic origin described above are utilized in the methods disclosed herein. In certain embodiment, the modified cells of leukemic origin comprising a mature dendritic cell phenotype are DCOne mDCs, which are off-the-shelf leukemic cell-dendritic cells. DCOne mDCs strongly express crucial T cell co-stimulatory ligands including CD40, CD70, CD80 and CD86. In addition, DCOne mDCs comprises at least one tumor antigen selected from the group consisting of WT-1, RHAMM, PRAME, MUC-1, p53, and Survivin, as described above. Therefore, the modified cells of leukemic origin can be directly utilized in the pre-REP phase without loading an additional antigen. Accordingly, in various embodiment, the methods disclosed herein do not require a further modification of the modified cells of leukemic origin in a process of loading an additional antigen on these cells. In one further embodiment, the pre-REP culture does not include any other antigen presenting cells, including other dendritic cells with or without loading specific antigens.

In certain embodiments, no additional cell feeders other than the described modified cells of leukemic origin described herein is employed in the pre-REP phase and added in the pre-REP culture.

The number of the modified cells of leukemic origin added at the beginning of the pre-REP expansion may vary, depending on the size of the tumor sample of the first population of TILs to be expanded. In certain embodiments, at the beginning of the pre-REP expansion, the cell ratio of the population of the modified cells of leukemic origin to the first population of TILs is from about 1:10 to about 1:1.

In various exemplary embodiments, the pre-REP expansion can be carried out for a period of about 7 days to about 21 days. For example, the first population of TILs and the population of modified cells of leukemic origin can be co-cultured during the pre-REP phase about 14 days or about 21 days.

In one exemplary embodiment, the REP expansion can be carried out for a period of about two weeks or less.

In various embodiments, IL-2 is typically present in the culture during the pre-REP phase at a high dose, such as a concentration ranging from about 1000 IU/mL to about 6000 IU/mL. In one exemplary embodiment, the concentration of IL-2 present in the culture during the pre-REP phase is about 6000 IU/mL.

In certain embodiments, the cytokines used in the REP expansion phase (the second phase) can be selected from IL-2, IL-15, IL-21, and/or IL-7. In one embodiment, IL-2 is utilized for the REP expansion. The cytokine are usually present at a high concentration. For instance, IL-2 may have a final concentration of about 1,000 IU/mL to about 6,000 IU/mL in the culture. In one embodiment, the culture during the REP phase comprises about 4000 IU/mL IL-2. In one embodiment, the culture during the REP phase comprises about 3000 IU/mL IL-2. In certain circumstance, during the REP phase, IL-2 may be added into the culture one or two days after the initiation of the REP phase.

In one embodiment, medium exchanges with fresh IL-2 are carried out regularly during the pre-REP phase and/or the REP phase to ensure continued T-cell division and survival during this time. In this situation, after each exchange, fresh IL-2 is added until a desired final concentration in the culture is achieved.

In one embodiment, TILs in the REP culture may be cultured and expanded for 14 days and then diluted as needed with 1:1 culture medium. IL-2 may be added in the new culture such that the final concentration of IL-2 is maintained as high as about 1,000 IU/mL to about 6,000 IU/mL.

In certain embodiments, the irradiated allogeneic PBMCs (feeder cells) utilized in the REP phase are obtained from at least three healthy donors. In one embodiment, the PBMCs are obtained from 4, 5, or 6 healthy donors. The number of the irradiated allogeneic PBMCs added into the culture at the beginning of the REP phase may be about 1-fold to about 200-fold of the number of co-cultured TILs from the second population of TILs (pre-TILs). In one embodiment, the number of the irradiated allogeneic PBMCs added into the culture at the beginning of the REP phase is about 200-fold of the number of co-cultured TILs from the second population of TILs (pre-TILs).

In certain embodiments during the REP phase, no additional types of antigen presenting cells (APCs) are added in the REP culture.

In certain embodiments, following the pre-REP phase and prior to the REP phase, tumor-specific TILs or TILs with superior tumor reactivity produced at the end of the pre-REP (pre-REP TILs) (the second population of TILs) are selected. Any selection method known in the art may be used. For instance, the selection may be performed by Ficoll density gradient centrifugation or via a specific cell sorting process. In one embodiment, dead cells are removed from the second population of the TILs prior to performing the REP expansion. In certain embodiments, TILs expanded from the pre-REP phase are not selected. Instead, the TILs from the second population of TILs are directly used as bulk TILs (young TILs) for expansion in the REP phase.

In certain embodiments, the pre-REP TILs are immediately cultured in the REP protocol culture medium for further TIL expansion. In certain embodiments, the pre-REP TILs are collected and cryopreserved after completion of the pre-REP expansion for a later REP expansion. Many freezing solutions and parameters are known in the art and will be useful in this context. In a non-limiting example, one method involves using PBS containing 20% DMSO and 8% human serum albumin, or other suitable cell freezing media. The cells are then frozen to −80° C. at a rate of about 1° C. per minute and stored in the vapor phase of a liquid nitrogen storage tank. Other methods of controlled freezing may be used as well as uncontrolled freezing immediately at −20° C. or in liquid nitrogen.

The method disclosed herein provides multiple advantages. For instance, as compared to a standard expansion process that comprises a standard pre-REP expansion without addition of the modified cells of leukemic origin in the culture and a standard REP expansion similar to the REP expansion described herein, the method disclosed herein provides at least one of the following advantages:
Significantly increase TIL number compared to the standard expansion protocol without addition of the modified cells of leukemic origin, e.g., DCOne mDC, in the pre-REP phase.
Despite a stronger expansion in the pre-REP phase, the expansion during the subsequent standard REP phase is also higher.
Preferential expansion of CD8+ T cells during the pre-REP phase that leads to a higher frequency of CD8+ T cells in the final product (after subsequent REP expansion).
Leads to nearly total disappearance of CD4+ and CD8+ T cells having the less differentiated CM phenotype while an increase in the total frequency of more differentiated effector-memory (EM) cells and terminally differentiated T_{EMRA} cells. In contrast, expansion in the standard pre-REP culture (without adding the modified cells of leukemic origin) resulted in a substantial amount of T cells, particularly CD4+ T cells, with the less differentiated CM phenotype.
A significantly lower terminal differentiation into T_{EMRA} cells.
A lower frequency of T cells expressing the exhaustion markers CD272 and PD-1 in the final TIL product (culture at the end of the REP phase).
An increased frequency of CD8+ T cells in the final product (14 days in the pre-REP phase+14 days in the REP phase) producing IFN-gamma upon activation with PHA, indicating a lower grade of exhaustion compared to CD8+ T cells from standard cultures without the presence of the modified cells of leukemic origin, e.g., DCOne mDC, in the pre-REP phase.

According to the present disclosure, addition of the modified cells of leukemic origin described herein can increase the initial IL-2-dependent proliferation of TILs during the pre-REP phase without negatively affecting the subsequent expansion during the standard REP phase and concomitantly avoid increased terminal differentiation and exhaustion of the expanded cells.

In certain exemplary embodiments, the third population of TILs produced according to the methods described above comprises a higher frequency of CD8+ T cells compared to the population of TILs produced by a process including a pre-REP expansion and a subsequent REP expansion in which the pre-REP expansion is performed without addition of the population of modified cells of leukemic origin.

In one exemplary embodiment, the third population of TILs produced according to the methods described above exhibits a decreased subpopulation of terminally differentiated effector memory T cells (T_{EMRA}) despite an increase of the frequency of differentiated effector-memory (EM) phenotype relative to a population of TILs produced by a process including a pre-REP expansion and a subsequent REP expansion in which the pre-REP expansion is performed without addition of the population of modified cells of leukemic origin.

In yet another exemplary embodiment, the obtained third population of TILs exhibits a lower grade of exhaustion as determined by a frequency of CD8+ T cells expressing PHA-induced IFN-γ, relative to a population of TILs produced by a process including a pre-REP expansion and a subsequent REP expansion in which the pre-REP expansion is performed without addition of the population of modified cells of leukemic origin.

In one embodiment, the method disclosed herein results in a greater expansion of TILs from the second population of TILs to the third population of TILs relative to a process including a pre-REP expansion and a subsequent REP expansion in which the pre-REP expansion is performed without addition of the population of modified cells of leukemic origin. In one embodiment, the third population of TILs is at least 1,000-fold greater in number than the second TIL population when the pre-REP expansion is performed for about 2 weeks and the REP expansion is performed for about 2 weeks.

In various embodiments, the second population of TILs obtained according to the above described method is greater in number than a population of TILs produced by culturing the first population of TILs in a pre-REP phase without addition of the population of modified cells of leukemic origin. In certain embodiments, the second population of TILs is more than 5-fold greater in number than the first population of TILs, i.e., TILs are expanded more than 5-fold at the completion of the first expansion in the pre-REP phase.

In various embodiments, the method results in a greater expansion of TILs from the first population of TILs to the third population of TILs relative to a process including a relative pre-REP expansion and subsequent REP expansion in which the relative pre-REP expansion is performed without addition of the population of modified cells of leukemic origin. In one embodiment, the third population of TILs is at least 5,000-fold greater in number than the first population of TILs when the pre-REP expansion is performed for about 2 weeks and the REP expansion is performed for about 2 weeks.

T cells generally constitute predominantly of the TILs in the cultures obtained at the completion of the pre-REP phase, as well as the REP phase. In one embodiment, the subpopulation of T cells constitutes greater than 60% of the total second population of TILs. In one embodiment, the subpopulation of T cells constitutes greater than 80% of the total second population of TILs when the pre-REP expansion is carried out for 14 days. In one embodiment, the subpopulation of T cells constitutes greater than 80% of the total second population of TILs when the pre-REP expansion is carried out for 14 days. In one embodiment, the subpopulation of T cells constitutes nearly 80% of the total second population of TILs when the pre-REP expansion is carried out for 14 days.

In one embodiment, the second population of TILs comprises an increased subpopulation of CD8+ T cells relative to a population of TILs produced by a pre-REP expansion process in which the first population of TILs is cultured without addition of the population of modified cells of leukemic origin. In one embodiment, the subpopulation of CD8+ T cells constitutes more than 30% of the total second population of TILs after the pre-REP expansion is performed for about 14 days.

In certain embodiments, method described above results in an increased ratio of the subpopulation of CD8+ T cells to the subpopulation of CD4+ T cells in the population of TILs obtained at the completion of the pre-REP phase, relative to the corresponding ratio resulted by a pre-REP expansion process in which the first population of TILs is cultured without addition of the population of modified cells of leukemic origin. In one embodiment, the cell ratio of the subpopulation of CD8+ T cells to the subpopulation of CD4+ T cells obtained at the end of the pre-REP expansion is more than 0.5 when the pre-REP expansion is performed for about 14 days. In another embodiment, the ratio of the subpopulation of CD 8+ T cells to the subpopulation of CD 4+ T cells in the second population of TILs is greater than 1.0 when the pre-REP expansion is performed for about 21 days.

In certain embodiments, the T cells within the second population of TILs exhibits a more differentiated phenotypic stage relative to a population of TILs produced by a pre-REP expansion process in which the first population of TILs is cultured without addition of the population of modified cells of leukemic origin.

In certain embodiments, the second population of TILs exhibits a decreased frequency of CD4+ and CD8+ T cells expressing the exhaustion markers CD272 and PD-1 determined by flow cytometry relative to a population of TILs produced by a pre-REP expansion process in which the first population of TILs is cultured without addition of the population of modified cells of leukemic origin.

In certain embodiments, the subpopulation of CD 8+ T_{EM} cells constitutes about 80% of the CD8+ T cells in the third population of TILs after performing the first expansion for about 14 days followed by the second expansion for about 14-days.

In certain embodiment, the subpopulation of CD 8+ T_{EMRA} cells constitutes about 10% of the CD8+ T cells in the third population of TILs after performing the first expansion for about 14 days followed by the second expansion for about 14 days, which is lower than the subpopulation of CD8+ T_{EMRA} in a population of TILs produced by a relative process including a relative first expansion for about 14 days and the second expansion for about 14 days in which the relative first expansion is performed without addition of the population of modified cells of leukemic origin.

In certain embodiment, the second population of TILs comprises an increased subpopulation of NKT cells relative to a population of TILs produced by a relative first expansion process in which the first population of TILs is cultured in the pre-REP culture medium without addition of the population of modified cells of leukemic origin.

In certain embodiments, methods disclosed herein further comprises a step of harvesting and sterilizing the third population of TILs produced at the end of the REP phase to produce a therapeutic population of TILs suitable for treating a cancer in a subject. TILs can be harvested in any appropriate and sterile manner, including for example by centrifugation. Methods for TIL harvesting are well known in the art and any such know methods can be employed with the present process. In some embodiments, TILs are harvested using an automated system.

In certain embodiments, the harvest, for example, is performed from a closed system bioreactor. In some embodiments, a closed system is employed for the TIL expansion and harvest. In certain embodiments, a single bioreactor is employed. In certain embodiment, the harvested and sterilized TILs are transferred to a container and formulated to be suitable for use in administration to a subject in need thereof. The therapeutic population of TILs is useful for adoptive cell therapy for treating cancer.

In certain embodiments, provided herein also includes a method for expanding tumor infiltrating lymphocytes (TILs) and producing a therapeutic population of TILs for treating cancer, comprising: (a) obtaining a tumor sample comprising a first population of TILs from a subject having a solid tumor cancer, (b) performing a pre-rapid expansion (pre-REP expansion) of TILs using a pre-rapid expansion protocol (pre-REP expansion) by co-culturing the first population of TILs with a population of modified cells of leukemic origin in a first culture comprising interleukin-2 (IL-2) for a period of time ranging from about 7 days to about 21 days, thereby producing a second population of TILs, wherein the population of modified cells of leukemic origin comprises a mature dendritic cell phenotype and is non-proliferating (e.g., via irradiation), and wherein the second population of TILs is greater in number than the first population of TILs; (c) performing a subsequent rapid expansion (RAP expansion) by culturing the second population of TILs produced in the first expansion in a rapid expansion protocol (REP) phase, without addition of the population of modified cells of leukemic origin, in a second culture comprising an anti-CD3 antibody, at least one cytokine, and irradiated allogeneic peripheral blood mononuclear cells (PBMCs) for 2 weeks or less, thereby producing a third population of TILs, wherein the third population of TILs is greater in number than the second population of TILs; and (d) harvesting and sterilizing the third population of TILs to produce a therapeutic population of TILs suitable for treating the cancer in the subject.

In certain embodiments, the therapeutic population of TILs generated according to the methods described above may comprise sufficient cell number of TILs for a therapeutically effective dosage of TILs for treating the cancer. In one embodiment, therapeutic population of TILs comprises from about 10 to about 250 billion TILs, which are sufficient as a therapeutically effective dosage for treating cancer. In one embodiment, therapeutic population of TILs comprises from about 10 to about 150 billion TILs.

### Expanding and Enriching Tumor Activating CD8+ T Cells From a Tumor Sample

In another aspect, provided herein is a method for expanding and enriching tumor activating CD8+ T cells from a tumor sample obtained from a subject having a particular tumor. As used herein, the term “enriching tumor activating CD8+ T cells” refers to increasing the proportion or population of functional CD8+ T lymphocytes in the TILs expanded from a tumor sample, where the tumor activating CD8+ T cells are capable of actively attacking and destroying cancer cells, thereby enhancing the body's anti-tumor immune response.

In various embodiment, the method for expanding and enriching tumor activating CD8+T cells comprises (a) obtaining a tumor sample comprising a population of primary TILs from a subject; (b) performing a pre-rapid expansion (pre-REP expansion) by co-culturing the tumor sample and a population of modified cells of leukemic origin using a pre-rapid expansion protocol (pre-REP) in the presence of interleukin-2 (IL-2), thereby producing a second population of TILs (pre-REP TILs), wherein the population of modified cells of leukemic origin comprises a mature dendritic cell phenotype and is non-proliferating (e.g., via irradiation), and wherein the second population of TILs is greater in number than the first population of TILs; and (c) performing a subsequent rapid expansion (REP expansion) of TILs by culturing the second population of TILs, without addition of the population of modified cells of leukemic origin, using a rapid expansion protocol (REP) in the presence of an anti-CD3 antibody, at least one cytokine, and irradiated allogeneic peripheral blood mononuclear cells (PBMCs), thereby producing a third population of TILs (REP TILs), wherein the third population of TILs is greater in number than the second population of TILs, and wherein the third population of TILs comprises a larger population of tumor activating CD8+ T cells relative to the population of tumor activating CD8+ T cells in a final population of TILs produced by a process including a pre-REP expansion and a subsequent REP expansion in which the pre-REP expansion is performed without addition of the population of modified cells of leukemic origin. In one embodiment, third population of TILs comprises a larger population of effector memory (T_{EM}) CD8+ T cells relative to the population of effector memory (T_{EM}) CD8+ T cells in a final population of TILs produced by a process including a pre-REP expansion and a subsequent REP expansion in which the pre-REP expansion is performed without addition of the population of modified cells of leukemic origin.

In certain embodiments, the above described methods further comprises a step of harvesting and sterilizing the third population of TILs produced at the end of the REP phase to produce a therapeutic population of TILs suitable for treating a cancer in a subject. TILs can be harvested in any appropriate and sterile manner, including for example by centrifugation. In certain embodiment, the above described method for expanding and enriching tumor activating CD8+ T cells further comprises a step of selecting the tumor activating CD8+ T cells from the third population of TILs. In certain embodiments, the method further comprises a step of sterilizing the selected tumor activating CD8+ T cells and formulating the selected cells to be suitable for cell therapy. In one embodiment, the pre-REP expansion is performed for a period of 2 weeks and the subsequent REP expansion is performed for a period of 2 weeks.

In certain embodiment, the tumor activating CD8+ T cells enriched according to the above described methods comprises enriched effector memory (T_{EM}) CD8+ T cells.

In one embodiment, the methods for enriching CD8+ T cells further comprise a step of selecting tumor-reactive CD8+ T cell for producing a final product of immune cells for adoptive cell therapy.

As to the methods for enriching CD8+ T cells described herein, various embodiments as to the TIL sources, the modified cells of leukemic origin, the feeder cells, cytokines added in the pre-REP and REP cultures, the durations of the pre-REP expansion and the REP expansion, as well as cell ratios, conditions, and/or results outline above are similarly comprised in the methods for enriching CD8+ T cells described herein.

### Use of the Modified Cells of Leukemic Origin in REP Phase

Disclosed herein further provides a new REP protocol using the modified cells of leukemic origin described here as feeder cells for expanding TILs in the rapid expansion phase, where the modified cells of leukemic origin comprise a mature dendritic cell phenotype and is non-proliferating or inactivated (e.g., via irradiation). The new REP protocol may be carried out following the pre-REP expansion phase described herein. The new REP protocol typically does not involve the use of PBMCs. In addition, anti-CD3 antibody is not required for the new REP protocol. Accordingly, in certain embodiments, the method for rapid expansion of TILs disclosed herein comprises culturing TILs in a modified rapid expansion culture comprising a population of the modified cells of leukemic origin described herein and a cytokine, where PBMCs are not included. In certain other embodiments, the rapid expansion of TILs is performed in a modified rapid expansion culture comprising a population of the modified cells of leukemic origin described herein and a cytokine, without adding any anti-CD3 antibodies, such as OKT-3. In another embodiment, the rapid expansion of TILs is performed in a modified rapid expansion culture comprising a population of the modified cells of leukemic origin described herein and a cytokine, without adding any anti-CD3 antibodies and PBMCs. In certain embodiments, the cell ratio of the modified cells of leukemic origin to the TILs added in the REP culture at the beginning of the REP expansion is from about 1:1 to about 10:1. In one embodiment, the cell ratio of the modified cells of leukemic origin to the TILs added in the REP culture at the beginning of the rep expansion is about 10:1.

Cytokines used in the new REP protocol are typically the same as described above for using in the standard REP protocol, which can be selected from IL-2, IL-15, IL-21, and/or IL-7. In one embodiment, IL-2 is utilized for the new REP expansion. The cytokine are usually present at a high concentration. For instance, IL-2 may have a final concentration of about 1,000 IU/mL to about 6,000 IU/mL in the culture. In one embodiment, the culture during the REP phase comprises about 4000 IU/mL IL-2. In one embodiment, the culture during the REP phase comprises about 3000 IU/mL IL-2. In certain circumstance, during the REP phase, IL-2 may be added into the new REP culture one or two days after the initiation of the REP phase. In one embodiment, the rapid expansion using the new REP protocol described herein is carried for about 14 days. In another embodiment, the rapid expansion using the new REP protocol described herein is carried for a period of less than 14 days.

In various embodiments, the new REP protocol can be used to replace the standard REP protocol used in the methods described above for ex vivo stimulation and expansion of TILs or for enriching CD8+ T cells described above. In certain embodiments, TILs expanded by the new REP protocol exhibit a higher proportion of effector function TILs, despite that the total expanded TILs at the end of the REP phase may be less in number, compared to the TILs expanded by the standard REP protocol where PBMCs are added as feeder cells.

For example, a method for ex vivo stimulation and expansion of TILs may comprise (a) performing a pre-rapid expansion (pre-REP expansion) of TILs by co-culturing a first population of TILs from a tumor sample and a first population of modified cells of leukemic origin in a pre-rapid expansion protocol (pre-REP) culture comprising interleukin-2 (IL-2), thereby producing a second population of TILs (pre-REP TILs), wherein the second population of TILs is greater in number than the first population of TILs; and (b) performing a subsequent rapid expansion (REP expansion) of TILs by culturing the second population of TILs in a rapid expansion protocol (REP) culture comprising a second population of modified cells of leukemic origin and at least one cytokine, without addition of any peripheral blood mononuclear cells (PBMCs), thereby producing a third population of TILs (REP TILs), wherein the third population of TILs is greater in number than the second population of TILs, and wherein the first population and the second population of the modified cells of leukemic origin comprises a mature dendritic cell phenotype and are non-proliferating (e.g., via irradiation). Optionally, an anti-CD3 antibody, such as OKT-3, is added in the REP culture. The cytokine are usually present at a high concentration. For instance, the cytokine may be IL-2 presented in the culture at a final concentration of about 1,000 IU/mL to about 6,000 IU/mL. The method may further comprises a step of (c) harvesting and sterilizing the third population of TILs to produce a therapeutic population of TILs suitable for treating the cancer in the subject.

In certain embodiments, the various exemplary methods described above further comprise a step of preparing the therapeutic population of TILs in a formulation suitable for delivering to a subject in need thereof to treat corresponding cancer.

In various embodiments, the therapeutic population of TILs may be used for cell therapy without further modification of the TILs.

In certain embodiments, the methods disclosed herein further comprise a process of modifying the TILs ex vivo before or after the pre-REP phase. In certain embodiments, the methods disclosed herein further include a process of modifying the TILs before or after REP phase. In one embodiment, TILs are genetically modified ex vivo to express a particular immune receptor or an particular immunomodulatory agent on the surface of the modified TILs. In certain embodiments, expanded TILs cells can be further modified ex vivo (e.g., genetically modified) to express a particular immune receptor (e.g., a chimeric antigen receptor (CAR)) binding to a particular antigen or marker expressed or displayed by the target tumor cells.

In various embodiments, the pre-REP expansion and the REP expansion as described herein may be performed in lab using any techniques known in the art. For instance, for the pre-REP expansion disclosed herein, digested tumor tissue or the first population of TILs described herein may be cultured with a population of modified cells of leukemic origin in a commercially available flasks with 100 cm gas-permeable silicon bottom that are commercially. TILs from a tumor sample may be suspended in a culture medium added with a population of modified cells of leukemic origin described herein in the presence of 1000 IU/mL to 6000 IU/mL IL-2, where the ratio of the number of the modified cells of leukemic origin added at the beginning of the pre-REP expansion in the culture to the number of primary TILs in the first population is from about 1:10 to about 1:1.

TILs expanded and produced according to the methods described above can be subsequently employed in a TIL therapy for treating cancer.

### Pharmaceutical Compositions and Formulations

Disclosed herein further provides pharmaceutical compositions and formulations, such as unit dose form compositions including the number of cells for administration in a given dose or fraction thereof for cell therapy.

In various embodiment, the pharmaceutical compositions disclosed herein comprise a therapeutic population of TILs produced according to the methods described herein. The TILs in the pharmaceutical composition have typically passed quality controls for sterility, negativity for blood-borne diseases, and phenotype checking. The pharmaceutical compositions and formulations generally include one or more optional pharmaceutically acceptable carrier or excipient. In certain embodiments, the compositions and formulations include at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In certain embodiments, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In certain embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in certain embodiments are included in the compositions. Suitable buffering agents include, for example, citric acid, sodium citrate, phosphoric acid, potassium phosphate, and various other acids and salts. In certain embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, e.g., those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. In certain embodiment, the pharmaceutical composition is formulated to include at least one unit dosage for treatment of cancer. Dose ranges and frequency of administration can vary depending on the nature of the population of the TILs produced by the methods described herein, the nature of the diseases to be treated, the medical condition of the subject to be treated, as well as the route of administration used. In some embodiments, a unit dosage includes from about 10 billion to about 250 billion TILs. In one embodiment, a unit dose includes from about 10 billion to about 150 billion TILs. The desired dosage can be delivered by a single bolus administration of the cells, by multiple bolus administrations of the cells, or by continuous infusion administration of the cells.

In certain embodiments, the pharmaceutical compositions can be formulated as suspensions and may contain other agents such as suspending, stabilizing, and/or dispersing agents. In one embodiment, TILs in the pharmaceutical composition are suspended in a sterile, balanced salt solution containing certain additional components, such as buffers, proteins (e.g., human serum albumin), cryoprotectants (for cryopreservation), and any other inactive excipients necessary to maintain cell viability and function during storage and administration.

In certain embodiments, the expanded TILs (including T cells) are formulated for parenteral administration. The term “parenteral,” as used herein, includes intravenous, intramuscular, subcutaneous, rectal, vaginal, and intraperitoneal administration. In certain embodiments, the cells are formulated for administration to the subject using peripheral systemic delivery by intravenous, intraperitoneal, or subcutaneous injection. In certain embodiments, the cells are formulated for bolus injection or continuous infusion.

Compositions in certain embodiments are provided as sterile liquid preparations, e.g., isotonic aqueous solutions, suspensions, emulsions, dispersions, or viscous compositions, which may in certain aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in certain aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### Treatment of Cancer With TILs

Disclosed herein also relates to employing the expanded TILs produced according to the methods disclosed herein in an adoptive cell therapy. Adoptive cell therapy is an immunotherapy in which immune cells (e.g., TILs cells, including CD8+ and CD4+ T cells) are given to a subject to fight diseases, such as cancer.

As such, In certain embodiments, disclosed herein is a method for treating a cancer or for preventing relapse of the cancer in a subject in need thereof comprising administering to the subject an effective dose of a population of TILs expanded and produced by the methods disclosed herein. In certain other embodiments, disclosed herein relates to methods for treating a cancer in a subject comprising administering to the subject a pharmaceutical composition comprising an effective dose of a therapeutic population of TILs produced by the methods disclosed herein.

In various embodiments, the treatment is autologous adoptive therapy, where TILs are ex vivo expanded from a tumor sample obtained from the subject having a cancer and administered back to the subject to treat the cancer.

In certain embodiments, an effective dose of a therapeutic population of TILs comprises from about 10 billion to about 250 billion TILs. In certain embodiments, the effective dose of the therapeutic population of TILs comprises from about 10 billion to about 150 billion TILs. In various embodiment, the therapeutic population of TILs may be administered to the subject by injection or infusion, or any other techniques commonly known in immunotherapy (see, e.g., Rosenberg et al., New Eng. J of Med. 1988, 319, 1676).

The optimal dosage and treatment regime for a particular subject can readily be determined by one skilled in the art (e.g., a doctor) according to the severity and course of the disease, whether the cells are administered for preventive or therapeutic purposes, previous therapy, the subject's clinical history and response to the cells, the weight of the subject, and the discretion of the attending physician. The compositions and cells are in some embodiments suitably administered to the subject at one time or over a series of treatments.

In various embodiments, the cancer to be treated is generally a solid tumor. Non-limiting examples of tumors to be treated by the methods described herein include, but are not limited to, melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and renal cell carcinoma. In one embodiment, the tumor to be treated is ovarian cancer. In another embodiment, the tumor to be treated is endometrial cancer.

In certain embodiments, the subject has been treated with a therapeutic agent targeting the disease or condition, e.g., the cancer, prior to administration of the TILs expanded according to the methods disclosed herein. In certain embodiments, the subject is refractory or non-responsive to the other therapeutic agent. In certain embodiments, the subject has persistent or relapsed disease, e.g., following treatment with another therapeutic intervention, including chemotherapy, radiation, and/or hematopoietic stem cell transplantation (HSCT), e.g., allogenic HSCT. In certain embodiments, the administration effectively treats the subject despite the subject having become resistant to another therapy. In certain embodiments, the subject receiving the treatment is in complete remission or partial remission after an initial treatment for the cancer, where the initial treatment may include a surgery to resect In certain embodiments, expanded TIL products that have passed the quality controls such as sterility, negativity for blood-borne diseases, and phenotype checking are prepared as a therapeutic population of TILs suitable to be administered to a subject in need thereof for treating a cancer. In certain embodiments, tumor activating CD8+ T cells are selected and administered to a subject for treating a cancer or preventing relapse of the cancer. In some embodiments, expanded TILs can also be used to treat a tumor that is benign.

In certain embodiments, prior to administration of the TILs, the subject receives lymphodepleting chemotherapy, where the subject is administered with an effective amount of an immunomodulatory agent. In certain embodiments, the lymphodepleting therapy is administrated with a non-myeloablative lymphodepletion regimen. The non-myeloablative lymphodepletion regimen can be commonly known in the art. In one embodiment, the non-myeloablative lymphodepletion regimen comprises administration of chemotherapy drugs, such as cyclophosphamide and/or fludarabine.

In certain embodiments, the methods for treating a cancer or preventing relapse of a cancer further described above comprises administering to the subject an effective amount of an immunomodulatory agent. In some embodiments, the immunomodulatory agent comprises a cytokine. In some embodiment, the cytokine is chosen from IL-2, IL-7, IL-15, IL-21, and variants or combinations thereof.

In certain embodiment when IL-2 is used, the first dose of IL-2 administration to the subject is about 3 hours to about 24 hours after the completion of TIL infusion at a dose of 600, 000 IU/kg intravenous every 8 to 12 hours. In one embodiment, the TILs from the therapeutic population of TILs described herein are applied up to a maximum of 6 doses.

In certain embodiments, a population of therapeutic TILs expanded and prepared by the methods disclosed herein are co-administered with an additional therapeutic agent that acts to elicit an immune response to a subject for treating or preventing a cancer. Nonlimiting examples of the additional therapeutic agent includes, but are not limited to, small molecule compounds, antibodies, or cellular reagents. The additional therapeutic and the TILs may be simultaneously or sequentially (in any order) administered to the subject. Suitable therapeutically effective dosages for each agent may be adjusted or lowered due to the additive action or synergy.

In certain embodiments, administration of the TILs or T cells of the disclosure may be combined with other methods useful to treat a disease or condition as determined by those of skill in the art.

### ILLUSTRATIVE EMBODIMENTS

The present disclosure is also described by the following illustrative embodiments.

Embodiment 1. A method for expanding tumor infiltrating lymphocytes (TILs), comprising:
(a) performing a first phase expansion of TILs by co-culturing a first population of TILs from a tumor sample and a population of modified cells of leukemic origin using a pre-rapid expansion protocol (pre-REP) in the presence of interleukin-2 (IL-2), thereby producing a second population of TILs, wherein the population of modified cells of leukemic origin comprises a mature dendritic cell phenotype and is non-proliferating, and wherein the second population of TILs is greater in number than the first population of TILs; and
(b) performing a second phase of expansion by culturing the second population of TILs using a rapid expansion protocol (REP) in the presence of an anti-CD3 antibody, at least one cytokine, and irradiated allogeneic peripheral blood mononuclear cells (PBMCs) without addition of the population of modified cells of leukemic origin, thereby producing a third population of TILs, wherein the third population of TILs is greater in number than the second population of TILs.

Embodiment 2. The method of embodiment 1, wherein the third population of TILs comprises a decreased subpopulation of terminally differentiated effector memory T cells (T_{EMRA}) despite an increase of the frequency of differentiated effector-memory (EM) phenotype relative to a population of TILs produced by a process including a pre-REP expansion and a subsequent REP expansion in which the pre-REP expansion is performed without addition of the population of modified cells of leukemic origin.

Embodiment 3. The method of embodiment 1 or 2, wherein the third population of TILs exhibits a lower grade of exhaustion relative to a population of TILs produced by a process including a pre-REP expansion and a subsequent REP expansion in which the pre-REP expansion is performed without addition of the population of modified cells of leukemic origin.

Embodiment 4. The method of any one of embodiments 1 to 3, wherein the method results in a greater expansion of TILs from the second population of TILs to the third population of TILs during the REP phase relative to a process including a pre-REP expansion and a subsequent REP expansion in which the pre-REP expansion is performed without addition of the population of modified cells of leukemic origin.

Embodiment 5. The method of any one of embodiments 1 to 4, wherein the third population of TILs is at least 1,000-fold greater in number than the second TIL population when the pre-REP expansion is performed for about 2 weeks and the REP expansion is performed for about 2 weeks.

Embodiment 6. The method of any one of embodiments 1 to 5, wherein the tumor sample is obtained from a tumor tissue of a subject having cancer, wherein the tumor tissue is from surgical resection, needle biopsy, core biopsy, small biopsy, or other means from a subject having cancer, and wherein the tumor sample contains a mixture of tumor cells and the first population of TILs.

Embodiment 7. The method of embodiment 6, further comprising a step of enzymatically digesting the tumor tissue prior to performing the first phase expansion, optionally wherein the tumor tissue is enzymatically digested by an enzyme chosen from collagenases, DNAse, and/or hyaluronidase.

Embodiment 8. The method of any one of embodiments 1 to 7, wherein the first population of TILs is a population of unselected TILs comprised in the tumor sample, and wherein the tumor sample is co-cultured with the population of modified cells of leukemic origin during the pre-REP phase in the presence of interleukin-2 (IL-2).

Embodiment 9. The method of any one of embodiment 1 to 8, wherein the tumor sample is from melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma.

Embodiment 10. The method of any one of embodiments 1 to 9, wherein the tumor sample is from ovarian or endometrial cancer.

Embodiment 11. The method of any one of embodiments 1 to 10, wherein the first phase expansion is performed for a period of about 7 days to about 21 days, optionally wherein the first phase expansion is performed for about 14 days or about 21 days.

Embodiment 12. The method of any one of embodiments 1 to 11, wherein the ratio of the number of the modified cells of leukemic origin added in the first phase expansion culture to the number of TILs in the first population is from about 1:10 to about 1:1.

Embodiment 13. The method of any one of embodiments 1 to 12, wherein IL-2 is present in the pre-REP phase culture at a concentration ranging from about 1000 IU/mL to about 6000 IU/mL.

Embodiment 14. The method of any one of embodiments 1 to 13, wherein IL-2 is present in the culture during the pre-REP phase at a concentration of about 6000 IU/mL.

Embodiment 15. The method of any one of embodiments 1 to 14, wherein the second population of TILs is greater in number than a population of TILs produced by culturing the first population of TILs in a pre-REP phase without addition of the population of modified cells of leukemic origin.

Embodiment 16. The method of any one of embodiments 1 to 15, wherein TILs are expanded more than 5-fold at the completion of the first expansion in the pre-REP phase.

Embodiment 17. The method of any one of embodiments 1 to 16, wherein the subpopulation of T cells constitutes greater than 60% of the total second population of TILs.

Embodiment 18. The method of any one of embodiments 1 to 17, wherein the second population of TILs comprises an increased subpopulation of CD8+ T cells relative to a population of TILs produced by a pre-REP expansion process in which the first population of TILs is cultured in the pre-REP phase without addition of the population of modified cells of leukemic origin.

Embodiment 19. The method of any one of embodiments 1 to 18, wherein the subpopulation of CD 8+ T cells constitutes more than 30% of the total second population of TILs after the pre-REP expansion is performed for about 14 days.

Embodiment 20. The method of any one of embodiments 1 to 19, wherein the second population of TILs comprises an increased ratio of the subpopulation of CD8+ T cells to the subpopulation of CD4+ T cells relative to a population of TILs produced by a pre-REP expansion process in which the first population of TILs is cultured without addition of the population of modified cells of leukemic origin.

Embodiment 21. The method of embodiment 20, wherein the cell ratio of the subpopulation of CD8+ T cells to the subpopulation of CD4+ T cells obtained at the end of the pre-RAP phase is more than 0.5 when the pre-REP expansion is performed for about 14 days.

Embodiment 22. The method of embodiment 20, wherein the cell ratio of the subpopulation of CD8+ T cells to the subpopulation of CD4+ T cells obtained at the end of the pre-REP phase is more than 1.0 when the pre-REP expansion is performed for about 21 days.

Embodiment 23. The method of any one of embodiments 1 to 22, wherein the second population of TILs exhibits a decreased frequency of CD4+ and CD8+ T cells expressing the exhaustion markers CD272 and PD-1 determined by flow cytometry relative to a population of TILs produced by a pre-REP phase expansion in which the first population of TILs is cultured without addition of the population of modified cells of leukemic origin.

Embodiment 24. The method of any one of claims 1 to 23, wherein the viability of the second population of TILs is greater than 80%.

Embodiment 25. The method of any one of embodiments 1 to 24, wherein the population of modified cells of leukemic origin comprises at least one tumor antigen selected from the group consisting of WT-1, RHAMM, PRAME, MUC-1, p53, and Survivin.

Embodiment 26. The method of any one of embodiments 1 to 25, wherein the population of modified cells of leukemic origin is CD34-positive, CD1a-positive, CD83-positive, and CD14-negative.

Embodiment 27. The method of any one of embodiments 1 to 26, wherein the population of modified cells of leukemic origin comprises a co-stimulatory molecule.

Embodiment 28. The method of any one of embodiments 1 to 27, wherein the population of modified cells of leukemic origin is CD40-positive, CD70-positive, CD80-positive, and CD86-positive.

Embodiment 29. The method of any one of embodiments 1 to 28, wherein the population of modified cells of leukemic origins is derived from cell line DCOne as deposited under the conditions of the Budapest treaty with the DSMZ under accession number DSMZ ACC 3189 on 15 Nov. 2012.

Embodiment 30. The method of any one of embodiments 1 to 29, wherein the irradiated allogeneic PBMCs are obtained from at least three healthy donors.

Embodiment 31. The method of any one of embodiments 1 to 30, wherein the number of the irradiated allogeneic PBMCs added into the culture at the start of the REP expansion is about 200-fold of the number of co-cultured TILs from the second population of TILs.

Embodiment 32. The method of any one of embodiments 1 to 31, wherein the at least one cytokine present in the culture during the REP phase is selected from IL-2, IL-15, IL-21, and/or IL-7.

Embodiment 33. The method of embodiment 32, wherein at least one cytokine comprises IL-2, and wherein the concentration of IL-2 in the REP phase is from about 1000 IU/mL to about 6000 IU/mL, optionally wherein the concentration of IL-2 in the REP phase is about 3000 IU/mL.

Embodiment 34. The method of any one of embodiments 1 to 33, wherein the second expansion is performed for a period of up to two weeks, optionally wherein the second expansion is performed for 14 days.

Embodiment 35. The method of any one of embodiments 1 to 34, wherein the method results in a greater expansion of TILs from the first population of TILs to the third population of TILs relative to a process including a relative pre-REP expansion and subsequent REP expansion in which the relative pre-REP expansion is performed without addition of the population of modified cells of leukemic origin.

Embodiment 36. The method of any one of embodiments 1 to 35, wherein the method results in an at least 5,000-fold expansion of TILs from the tumor sample after the pre-REP expansion is performed for about 2 weeks and the REP expansion is performed for about 2 weeks.

Embodiment 37. The method of any one of embodiments 1 to 36, wherein the subpopulation of CD8+ T_{EM} cells constitutes about 80% of the CD8+ T cells in the third population of TILs after performing the first expansion for about 14 days followed by the second expansion for about 14-days.

Embodiment 38. The method of any one of embodiments 1 to 37, wherein the subpopulation of CD8+ T_{EMRA} cells constitutes about 10% of the CD8+ T cells in the third population of TILs after the first expansion is performed for 14 days and the subsequent second expansion is performed for about 14 days, which is lower than the subpopulation of CD8+ T_{EMRA} in a population of TILs produced by a relative process including a relative first expansion for about 14 days and the second expansion for about 14 days in which the relative first expansion is performed without addition of the population of modified cells of leukemic origin.

Embodiment 39. The method of any one of embodiments 1 to 37, wherein the subpopulation of CD8+ T_{EMRA} cells constitutes about 20% of the CD8+ T cells in the third population of TILs after performing the first expansion for about 21 days followed by the second expansion for about 14 days, where the subpopulation of CD8+ T_{EMRA} is smaller compared to a corresponding subpopulation of CD8+ T_{EMRA} produced by a process including a relative pre-REP expansion and subsequent REP expansion in which the relative pre-REP expansion is performed without addition of the population of modified cells of leukemic origin.

Embodiment 40. The method of any one of embodiments 1 to 39, wherein the method results in a lower frequency of T cells expressing exhaustion markers CD272 and PD-1 in the third population of TILs relative to a process including a relative first expansion and the second expansion in which the relative first expansion is performed without addition of the population of modified cells of leukemic origin.

Embodiment 41. The method of any one of embodiments 1 to 40, wherein the third population of TILs exhibits a lower grade of exhaustion of CD8+ T cells as measured by an increased frequency of CD8+ T cells producing IFNγ upon activation with PHA, when the pre-REP phase and the REP phase are respectively performed for 14 days for each phase, relative to a population of TILs produced by a process including a pre-REP expansion and a subsequent REP expansion in which the pre-REP expansion is performed without addition of the population of modified cells of leukemic origin.

Embodiment 42. The method of any one of embodiments 1 to 41, further comprising a step of harvesting and sterilizing the third population of TILs to produce a therapeutic population of TILs suitable for treating a cancer.

Embodiment 43. The method of embodiment 42, wherein the therapeutic population of TILs comprises sufficient cell number of TILs for effectively treating the cancer.

Embodiment 44. The method of embodiment 43, wherein the cell number of TILs sufficient for an effective dose is from about 10 billion to about 250 billion, optionally from about 10 million to about 150 billion.

Embodiment 45. A method for expanding tumor infiltrating lymphocytes (TILs) and producing a therapeutic population of TILs for treating cancer, comprising:
(a) obtaining a tumor sample comprising a first population of TILs from a donor subject having cancer;
(b) performing a first expansion of TILs by co-culturing the first population of TILs and a population of modified cells of leukemic origin during a pre-REP phase in a culture comprising interleukin-2 (IL-2) for a first period of time ranging from about 7 days to about 21 days, thereby producing a second population of TILs,
   wherein the population of modified cells of leukemic origin comprises a mature dendritic cell phenotype and is non-proliferating, and
   wherein the second population of TILs is greater in number than the first population of TILs;
(c) performing a second expansion by culturing the second population of TILs in a REP phase in a culture comprising an anti-CD3 antibody, at least one cytokine, and irradiated allogeneic peripheral blood mononuclear cells (PBMCs) without addition of the population of modified cells of leukemic origin for a second period of up to 2 weeks, thereby producing a third population of TILs, wherein the third population of TILs is greater in number than the second population of TILs; and
(d) harvesting and sterilizing the third population of TILs to produce a therapeutic population of TILs suitable for treating a cancer in a receiver subject in need thereof.

Embodiment 46. The method of embodiment 45, wherein the donor subject is the receiver subject, and wherein the therapeutic population of TILs is for autologous treatment.

Embodiment 47. A method for expanding and enriching tumor activating CD8+ T cells comprising:
(a) Obtaining a tumor sample comprising a population of primary TILs from the cancer donor;
(b) performing a pre-rapid expansion by co-culturing the tumor sample and a population of modified cells of leukemic origin in a pre-rapid expansion protocol (pre-REP) culture comprising interleukin-2 (IL-2), thereby producing a second population of TILs (pre-REP TILS),
wherein the population of modified cells of leukemic origin comprises a mature dendritic cell phenotype and is non-proliferating, and
wherein the second population of TILs is greater in number than the first population of TILs; and
(c) performing a rapid expansion of TILs by culturing the second population of TILs in a rapid expansion protocol (REP) culture comprising an anti-CD3 antibody, at least one cytokine, and irradiated allogeneic peripheral blood mononuclear cells (PBMCs), without addition of the population of modified cells of leukemic origin, thereby producing a third population of TILs (REP TILs), wherein the third population of TILs is greater in number than the second population of TILs, wherein the third population of TILs comprises a larger subpopulation of tumor activating CD8+ T cells relative to the subpopulation of tumor activating CD8+ T cells produced by a process including a pre-REP expansion and a subsequent REP expansion in which the pre-REP expansion is performed without addition of the population of modified cells of leukemic origin.

Embodiment 48. The method of embodiment 47, wherein the pre-REP expansion is performed for 1, 2, or 3 weeks, and/or the REP expansion is performed for 2 weeks.

Embodiment 49. The method of embodiment 47 or 48, further comprises selecting tumor activating CD8+ T cells from the third population of TILs to prepare a therapeutic population of cells enriched for tumor-reactive T cells.

Embodiment 50. A pharmaceutical composition comprising a therapeutic population of therapeutic tumor infiltrating lymphocytes (TILs) produced according to any one of the method of embodiments 42 to 46, or a therapeutic population of tumor activating CD8+ cells produced according to embodiment 49.

Embodiment 51. A method for treating a subject having cancer, comprising administering an effective dose of the therapeutic population of TILs produced by the method according to any one of embodiments 42 to 46, a therapeutic population of tumor activating CD8+cells produced according to embodiment 49, or the pharmaceutical composition of Embodiment 50.

Embodiment 52. The method of embodiment 51, wherein the effective dose of the therapeutic population of TILs comprises from about 10 billion to about 250 billion TILs, optionally wherein the effective dose comprise from about 10 billion to about 150 billion TILs.

Embodiment 53. The method of embodiment 51 or 52, further comprises administering to the subject an effective amount of an immunomodulatory agent prior to the administration of the therapeutic population of TILs.

Embodiment 54. The method of embodiment 53, wherein the immunomodulatory agent comprises a cytokine.

Embodiment 55. The method of embodiment 54, wherein the cytokine is chosen from IL-2, IL-7, IL-15, IL-21, and variants or combinations thereof.

Embodiment 56. A method for expanding tumor infiltrating lymphocytes (TILs) from a tumor sample, comprising co-culturing an enzymatically digested tumor sample prepared from tumor tissue of a subject and a population of modified cells of leukemic origin in a pre-rapid expansion protocol (pre-REP) culture comprising IL-2 for a period of about 7 days to about 21 days, thereby producing a population of pre-REP TILs expanded from the tumor sample;

wherein the population of modified cells of leukemic origin population comprises a mature dendritic cell phenotype and is non-proliferating; and
wherein IL-2 is present in the pre-REP culture at a concentration ranging from about 1000 IU/mL to about 6000 IU/mL.

Embodiment 57. The method of embodiment 56, where the cell ratio of the population of the modified cells of leukemic origin added in the pre-REP culture at the beginning of the pre-rapid expansion, to the cells from enzymatically digested tumor sample is from about 1:10 to about 1:1.

Embodiment 58. The method of embodiment 46 or 47, further comprising a rapid expansion by culturing the population of pre-REP TILs produced at the end of the first expansion in a rapid expansion protocol (REP) culture comprising anti-CD3 antibodies, at least one cytokine, and irradiated allogeneic peripheral blood mononuclear cells (PBMCs) without addition of the population of modified cells of leukemic origin, thereby producing a population of REP TILs, wherein the subpopulation of REP TILs is greater in number than the subpopulation of pre-REP TILs.

Embodiment 59. A method for ex vivo stimulation and expansion of tumor infiltrating lymphocytes (TILs) or enriching CD8+ T cells from a tumor sample, comprising:
performing a pre-rapid expansion of TILs by co-culturing a first population of TILs from a tumor sample and a population of modified cells of leukemic origin in a pre-rapid expansion protocol (pre-REP) culture comprising interleukin-2 (IL-2), thereby producing a second population of TILs,
wherein the population of modified cells of leukemic origin comprises a mature dendritic cell phenotype and is non-proliferating, and
wherein the second population of TILs is greater in number than the first population of TILs.

Embodiment 60. The method of embodiment 59, wherein the pre-rapid expansion is performed for a period of about 7 days to about 21 days, optionally about 14 days or 21 days.

Embodiment 61. The method of embodiment 59 or 60, wherein the ratio of the number of the modified cells of leukemic origin added in the first phase expansion culture to the number of TILs in the first population is from about 1:10 to about 1:1.

Embodiment 62. The method of any one of embodiments 59 to 61, wherein IL-2 is present in the pre-REP culture at a concentration ranging from about 1000 IU/mL to about 6000 IU/mL.

Embodiment 63. The method of any one of embodiments 59 to 62, wherein the first population of TILs is a population of unselected TILs comprised in the tumor sample, and wherein the tumor sample is co-cultured with the population of modified cells of leukemic origin in the pre-REP culture.

Embodiment 64. The method of any one of embodiments 59 to 63, wherein TILs are expanded more than 5-fold at the completion of the pre-rapid expansion.

Embodiment 65. The method of any one of embodiments 59 to 64, wherein the tumor sample is from melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma.

Embodiment 66. The method of any one of embodiments 59 to 65, wherein the second population of TILs is greater in number than a population of TILs produced by culturing the first population of TILs in a pre-REP phase without addition of the population of modified cells of leukemic origin.

Embodiment 67. The method of any one of embodiments 59 to 66, wherein the second population of TILs comprises an increased subpopulation of CD8+ T cells relative to a population of TILs produced by a pre-REP expansion process in which the first population of TILs is cultured in the pre-REP phase without addition of the population of modified cells of leukemic origin.

Embodiment 68. The method of any one of embodiments 59 to 67, wherein the second population of TILs comprises an increased ratio of the subpopulation of CD8+ T cells to the subpopulation of CD4+ T cells relative to a population of TILs produced by a pre-REP expansion process in which the first population of TILs is cultured without addition of the population of modified cells of leukemic origin.

Embodiment 69. The method of any one of embodiments 59 to 68, wherein the population of modified cells of leukemic origin comprises:
at least one tumor antigen selected from the group consisting of WT-1, RHAMM, PRAME, MUC-1, p53, and Survivin;
is CD34-positive, CD1a-positive, CD83-positive, and CD14-negative; and/or
is CD40-positive, CD70-positive, CD80-positive, and CD86-positive.

Embodiment 70. The method of any one of embodiments 59 to 69, wherein the population of modified cells of leukemic origins is derived from cell line DCOne as deposited under the conditions of the Budapest treaty with the DSMZ under accession number DSMZ ACC 3189 on 15 Nov. 2012.

### Embodiment 71. the method of any one of embodiments 59 to 70, further comprising:

performing a subsequent rapid expansion of TILs by culturing the second population of TILs in a rapid expansion protocol (REP) culture comprising at least one cytokine, without addition of peripheral blood mononuclear cells (PBMCs), thereby producing a third population of TILs, wherein the third population of TILs is greater in number than the second population of TILs.

Embodiment 72. the method of any one of embodiments 59 to 71, further comprising:
performing a subsequent rapid expansion of TILs by culturing the second population of TILs in a rapid expansion protocol (REP) culture comprising at least one cytokine, without addition of any anti-CD3 antibody, thereby producing a third population of TILs, wherein the third population of TILs is greater in number than the second population of TILs.

Embodiment 73. The method of embodiment 71 or 72, wherein the at least one cytokine present in the culture during the REP phase is selected from IL-2, IL-15, IL-21, and/or IL-7.

Embodiment 74. The method of embodiment 73, wherein at least one cytokine comprises IL-2, and wherein the concentration of IL-2 in the REP phase is from about 1000 IU/mL to about 6000 IU/mL, optionally wherein the concentration of IL-2 in the REP phase is about 3000 IU/mL.

Embodiment 75. The method of any one of embodiments 71 to 74, wherein the rapid expansion is performed for a period of up to two weeks, optionally wherein the rapid expansion is performed for 14 days.

Embodiment 76. The method of any one of embodiments 59 to 70, further comprising a step of harvesting and sterilizing the second population of TILs to produce a therapeutic population of TILs suitable for treating a cancer in a receiver subject in need thereof.

Embodiment 77. The method of any one of embodiments 71 to 75, further comprising a step of harvesting and sterilizing the third population of TILs to produce a therapeutic population of TILs suitable for treating a cancer in a receiver subject in need thereof.

Embodiment 78. A pharmaceutical composition comprising a therapeutic population of therapeutic tumor infiltrating lymphocytes (TILs) produced according to embodiment 76 or 77.

Embodiment 79. A method for treating a subject having cancer, comprising administering an effective dose of the therapeutic population of TILs produced by the method according to embodiment 76 or 77.

Embodiment 80. The method of embodiment 79, wherein the effective dose of the therapeutic population of TILs comprises from about 10 billion to about 250 billion TILs, optionally wherein the effective dose comprise from about 10 billion to about 150 billion TILs.

Embodiment 81. The method of embodiment 79 or 80, further comprises administering to the subject an effective amount of an immunomodulatory agent prior to the administration of the therapeutic population of TILs.

### Embodiment 82. the method of embodiment 82, wherein the immunomodulatory agent comprises a cytokine.

Embodiment 83. The method of embodiment 82, wherein the cytokine is chosen from IL-2, IL-7, IL-15, IL-21, and variants or combinations thereof.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples further illustrate aspects of the present disclosure. However, it is in no way a limitation of the teachings of the present disclosure as set forth. It should be understood that these examples are given by way of illustration only. From the above discussion and these examples, one of ordinary skill in the art can ascertain the essential characteristics of embodiments of the present disclosure. Without departing from the spirit and scope thereof, one skilled in the art can make various changes and modifications of the disclosure to adapt it to various usages and conditions. All publications, including patents and non-patent literature, referred to in this specification are expressly incorporated by reference herein.

In the following examples, studies were performed to investigate if addition of mature dendritic cells derived from the DCOne leukemic cell line (DCOne mDCs) would increase the initial IL-2-dependent proliferation of TILs without negatively affecting the subsequent rapid expansion during the standard REP phase, and concomitantly avoid increased terminal differentiation and exhaustion of the expanded cells.

mDCs may be obtained by the method described in U.S. Pat. No. 10,064,923: (1) first culturing DCOne progenitor cells in routine maintenance medium consisting of MEM-α (Minimum essential medium, Lonza, Verviers, Belgium) containing 10% fetal calf serum (FCS) (Hyclone, Perbio Science, Etten-Leur, The Netherlands), 100 IU/ml sodium-penicillin (pen), 100 μg/ml streptomycin (strep), 2 mM L-glutamine (glut), 50 μM β-mercaptoethanol (2ME) and GM-CSF (5 ng/ml); (2) allowing the DCOne progenitor cells to differentiate into immature DCOne for 6 days by adding 1000 IU/ml GM-CSF, 20 ng/ml IL-4 and 120 IU/ml TNF-α, where fresh cytokines were added on day 3; and (3) inducing maturation by adding mimic mix (2400 IU/ml TNF-α, 100 ng/ml IL-6, 1 ug/ml PGE 2 and 25 ng/ml IL 1-β) for 2 days.

### Example 1: Addition of DCOne mDC to the Standard Pre-rapid Expansion Protocol (pre-REP) Culture Results in Superior Expansion of Tumor Infiltrating Lymphocytes

In this example, a study was performed to investigate if addition of DCOne mDC during the pre-rapid expansion phase would benefit ex vivo expanding TILs from tumor samples. In this study, tumor dissociates from four ovarian cancer patient donors and five endometrial cancer patient donors were respectively cultured for 14 days to 21 days in the standard pre-rapid expansion protocol (pre-REP) culture comprising 6000 IU/mL high IL-2, with or without addition of irradiated DCOne mDCs.

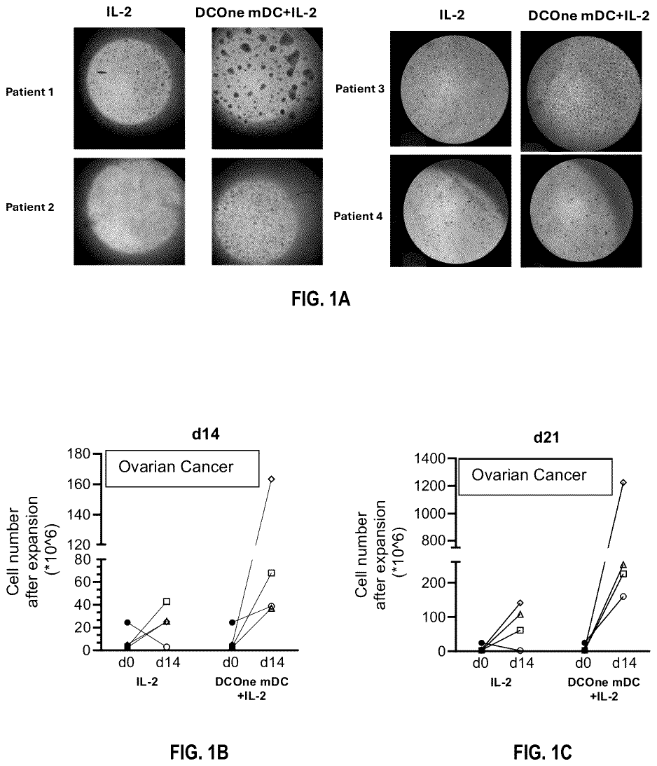

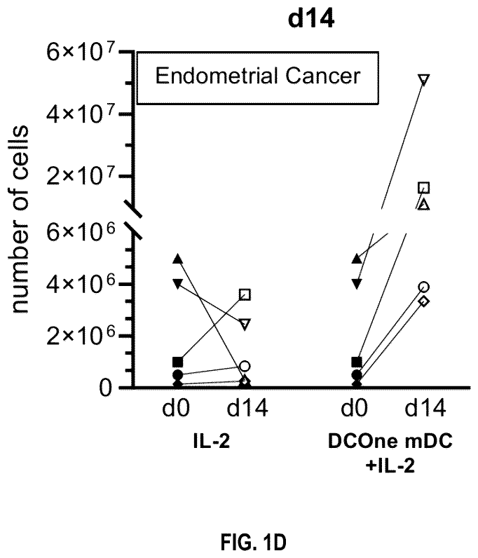

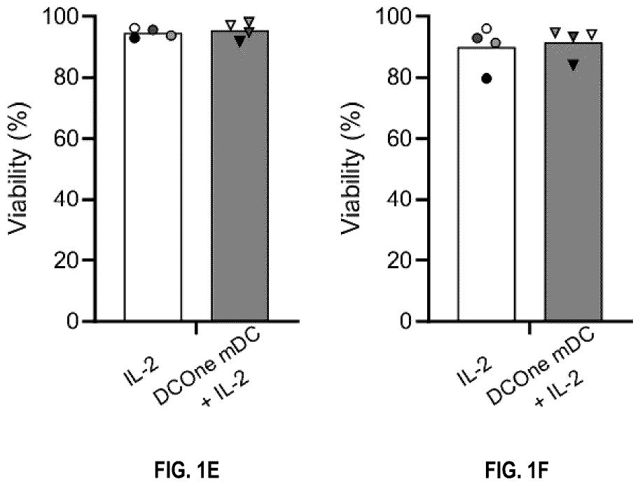

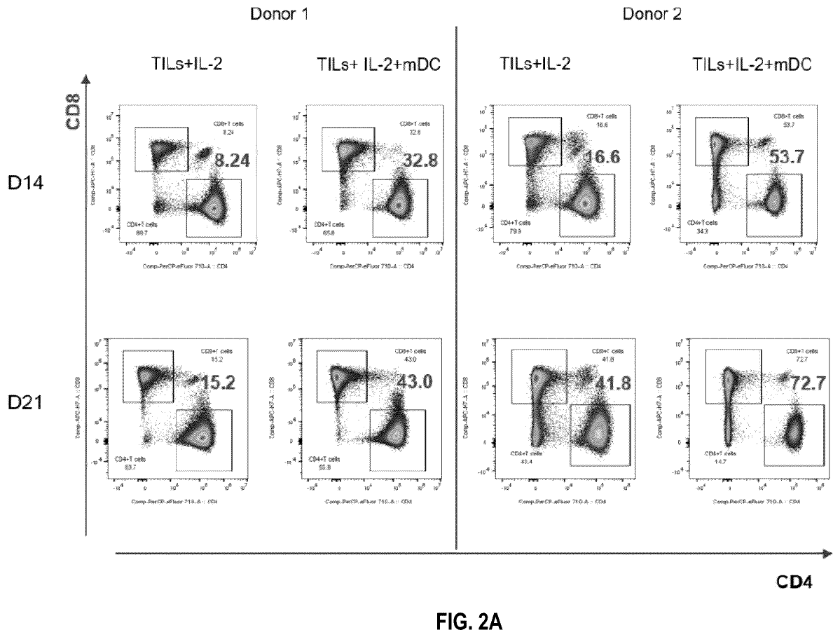

FIG. 1A depicts the light microscope cell culture representative images of the ovarian cancer TIL cultures on day 7 during the pre-REP phase, where cultures with or without DCOne mDC are compared.

On day 14 and day 21 from the start of the pre-REP culturing, cells were harvested from the pre-REP cultures and cell numbers were counted to investigate the pre-REP expansion of TILs. 1B and 1C graphically depicts the pre-REP expansion of ovarian cancer TILs for 14 days (FIGS. 1B) and 21 days (FIG. 1C) in pre-REP cultures in the presence of 6000 IU/mL IL-2, with or without DCOne mDC. FIG. 1D depicts the pre-REP expansion of endometrial cancer TILs for 14 days in the pre-REP cultures in the presence of 6000 IU/mL IL-2, with or without addition of DCOne mDC. As shown in FIGS. 1B, 1C, and 1D, addition of DCOne mDC to the standard pre-rapid expansion protocol (pre-REP) culture results in superior expansion of TILs in the pre-REP phase, compared to expansion of TILs in the standard pre-REP culture without added DCOne mDC.

FIGS. 1E and 1F graphically depicts the cell viabilities of TILs from ovarian cancer patients as measured using nucleocounter on day 14 (FIG. 1E) and day 21 (FIG. 1F) of the pre-REP culture. Results show high viability (greater than 80%) of TILs expanded from tumor dissociates of ovarian cancer when the TILs were co-cultured with DCOne mDCs in the presence of IL-2 for 14 days (FIGS. 1E) and 21 days (FIG. 1F). Compared to culturing the TILs in the standard pre-REP culture that does not include DCOne mDCs, co-culturing of TILs and DCOne mDCs in the pre-REP culture does not negatively affect the viability of expanded TILs.

### Example 2: Immune Cell Subsets After 14 and 21 Days in the pre-REP Phase With and Without Addition of DCOne mDC

A study was performed to analyze Immune cell subsets after culturing tumor dissociates from ovarian cancer patients using the standard pre-REP culture comprising 6000 IU/mL IL-2, with or without addition of DCOne mDCs.

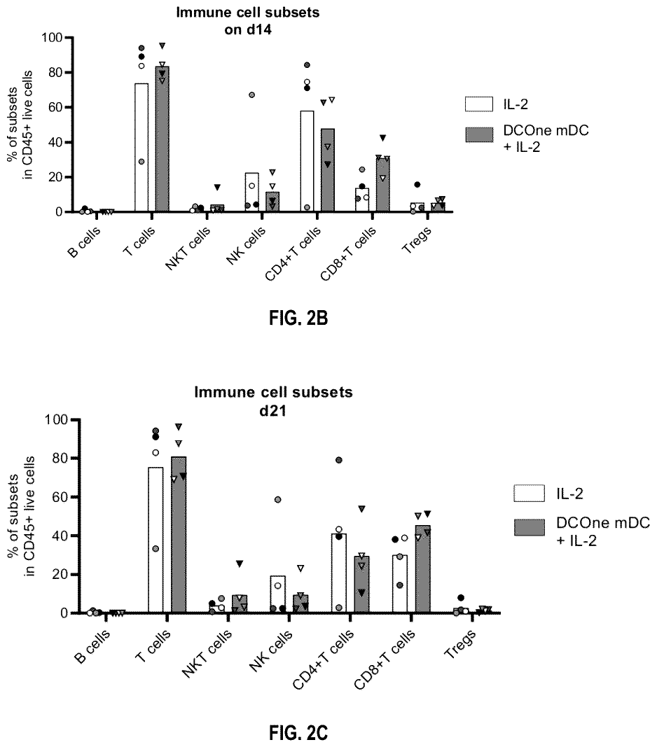

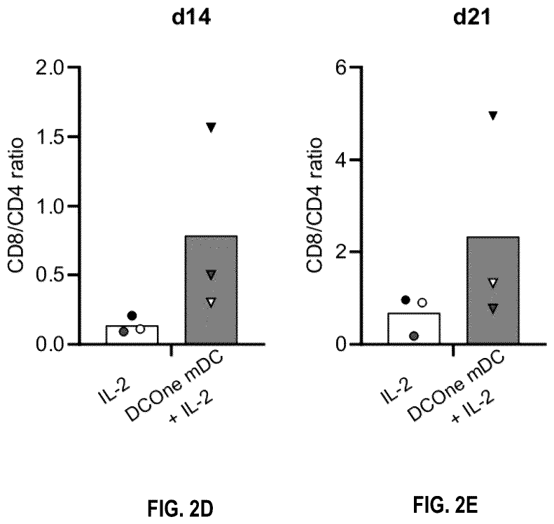

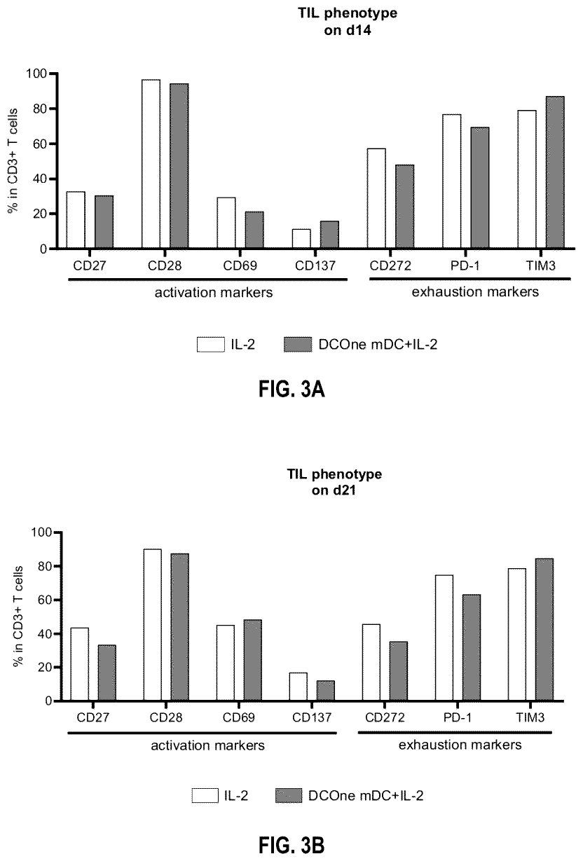

On day 14 and day 21 after starting the pre-REP culturing, cells in the pre-REP cultures were analyzed and compared using a flow cytometer. FIG. 2A depicts representative flow cytometric plots of CD4+ T cells and CD8+ T cells in pre-REP cultures of TILs from two ovarian cancer donors, with or without DCOne mDCs stimulation. As shown in FIG. 2A, co-culturing TILs with DCOne mDCs in the pre-REP culture comprising IL-2 resulted in a significantly higher proportion of CD8+ T cells, compared to TILs cultured in the pre-REP culture without added DCOne mDCs, on day 14 and day 21 in the pre-REP phase.

Cells from the day 14 and day 17 cultures were also harvested, stained with fluorochrome labelled cell surface molecule specific antibodies, and analyzed to investigate immune cell subsets (including B cells, T cells, NKT cells, NK cells, CD4+ T cells, CD8+ T cells, and Regulatory T (Treg) cells) using flow cytometry. Live immune cells (TILs) were identified as CD45+. The results are shown in FIGS. 2B to 2D.

FIGS. 2B and 2C graphically depict the immune cell composition of day 14 (FIG. 2B) and day 21 (FIG. 2C) pre-REP cultures of TILs from the ovarian cancer donors. As shown in FIGS. 2B and 2C, T cells constituted a predominant proportion in the total CD45+ live cells after expanding TILs in the pre-REP cultures for 14 and 21 days. The proportion of total T cells was higher in the pre-REP TIL culture added with DCOne mDC, compared to the pre-REP TIL culture not added with DCOne mDC. In addition, the proportion of CD8+ T cells in the pre-REP culture added with DCOne mDC was significantly higher (more than 30% on day 14 and more than 40% on day 21) than the corresponding proportion of CD8+ T cells in the pre-REP culture without DCOne mDCs (less than 20% on day 14 and less than 40% on day 21).

The cell ratios of CD8+ T cells to CD4+ T cells in the cultures on day 14 and day 21 of the pre-REP phase were further analyzed. The results show that the cell ratio of CD8+ T cells to CD4+ T cells (CD8:CD4 ratio) was significantly higher in the pre-REP cultures added with DCOne mDCs, compared to the pre-REP cultures without addition of DCOne mDCs. As depicted in FIG. 2D, on day 14 of the pre-REP phase, the CD8:CD4 ratio was greater than 0.5 in the co-cultures of TILs and DCOne mDCs, while the CD8:CD4 ratio was less than 0.2 in the cultures of TILs without DCOne mDCs. Also, as shown in FIG. 2E, on day 21 of the pre-REP phase, the CD8:CD4 ratio was greater than 2 in the co-cultures of TILs and DCOne mDCs, while the CD8:CD4 ratio was less than 1 in the cultures of TILs without DCOne mDCs.

These results show that CD8+ T cells are preferentially expanded in the pre-REP phase when the standard pre-REP culture is added with DCOne mDCs.

### Example 3: TIL Phenotypes After 14 and 21 Days of Culture Using the Standard pre-REP Protocol With or Without Addition of DCOne mDCs

TIL phenotypes after 14 and 21 days of culturing using that standard pre-REP protocol with or without addition of DCOne mDCs were investigated. Tumor samples used in this study were from ovarian cancer donors. Phenotypes were determined by flow cytometry.

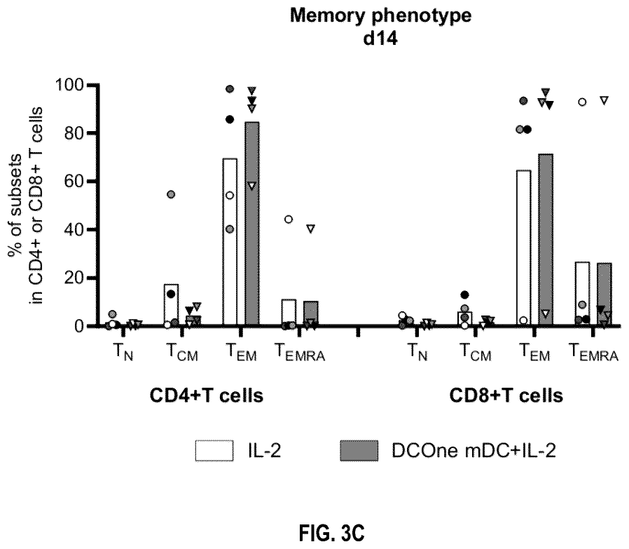

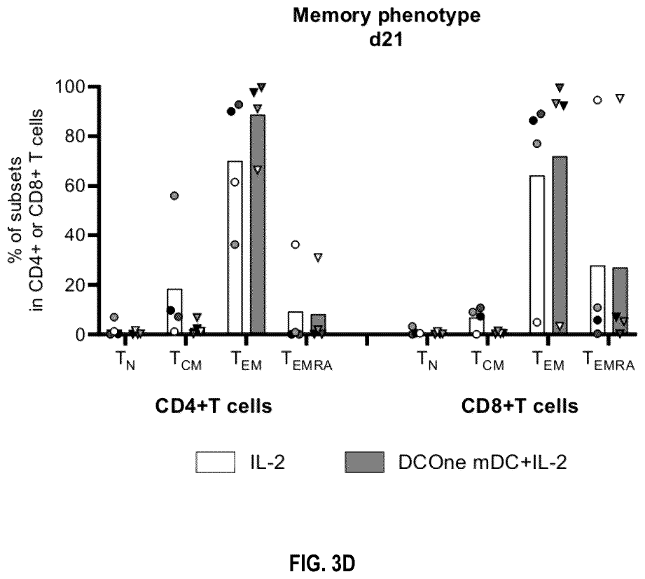

FIGS. 3A and 3B depict the expression of activation and checkpoint molecules on CD3 T cells in day 14 (FIG. 3A) and day 21 (FIG. 3B) pre-REP cultures, where the cultures with or without the addition of DCOne mDCs are compared. The results show a decreased frequency of CD4+ and CD8+ T cells expressing the exhaustion markers CD272 and PD-1 in the pre-REP phase, when the TILs were co-cultured with DCOne mDCs in the presence of IL-2, compared to when the TILs were cultured in the presence of IL-2 without added DCOne mDCs.

FIGS. 3C and 3D depicts the frequencies of CD4 and CD8 naive (T_{N}), central memory (T_{CM}), effector memory (T_{EM}), and Terminally differentiated effector memory (T_{EMRA}) T cells in the cultures with or without DCOne mDCs on day 14 (FIG. 3C) and day 21 (FIG. 3D) of the pre-REP phase. Phenotypic analysis with flow cytometry showed that after expansion TILs in the standard pre-REP culture with addition of DCOne mDC for 14 or 21 days, CD4+ and CD8+ T cells with the less differentiated central-memory (CM) phenotype were nearly total disappeared. In contrast, expansion in the standard pre-REP culture without added DCOne mDC resulted in a substantial proportion of T cells, particularly CD4+ T cells, with the less differentiated CM phenotype. In addition, the total frequency of more differentiated effector-memory (EM) cells and terminally differentiated T_{EMRA} cells increased in the pre-REP cultures added with DCOne mDCs, compared to the pre-cultures without any DCOne mDCs.

### Example 4: Expansion Level and Phenotypes After TILs Are Expanded in the pre-REP Phase With or Without Added DCOne mDCs for 14 Days or 21 Days and Subsequently Expanded in the REP Phase for 14 Days

A study was performed to investigate if addition of DCOne mDCs during the pre-REP phase would affect the subsequent expansion of TILs during the standard REP phase. TILs expanded in the above two different pre-REP cultures (with or without DCOne mDCs) for 14 days or 21 days as described in Example 3 were subsequently expanded for 14 days using the standard REP protocol in the presence of irradiated PBMC from 3 different healthy donor, IL-2 (3000 IU/mL), and anti-CD3 antibody (OKT-3 clone), without adding any DCOne mDCs. At the completion of the REP expansion, cells from the final REP TIL cultures were counted and harvested, stained with fluorochrome labelled cell surface molecule specific antibodies, and analyzed with flow cytometry to investigate TIL expansion and phenotype.

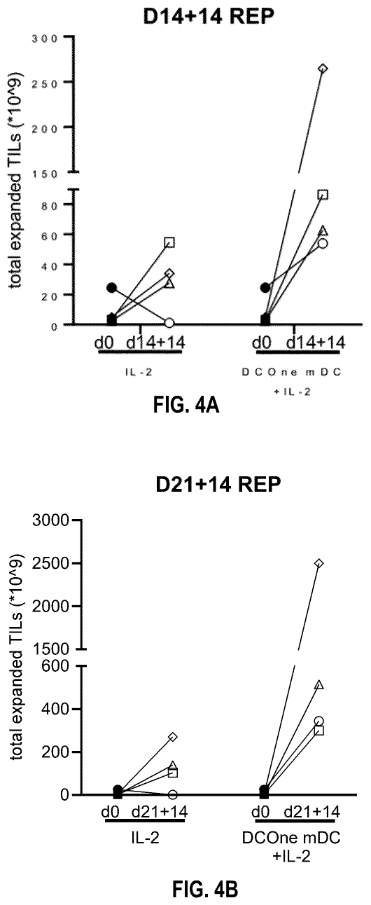

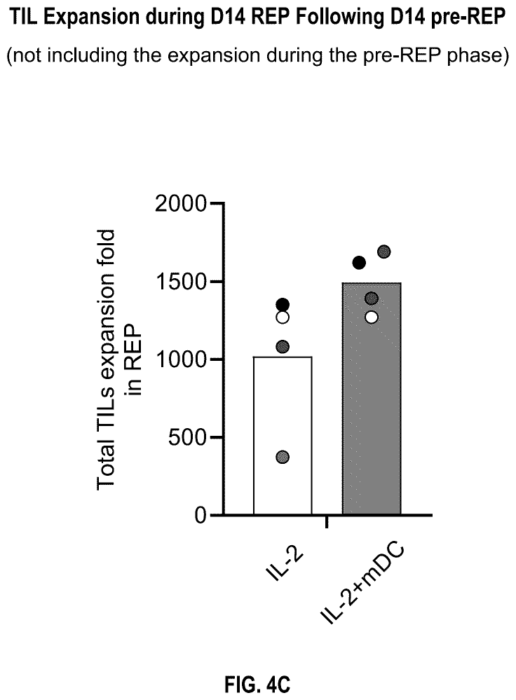

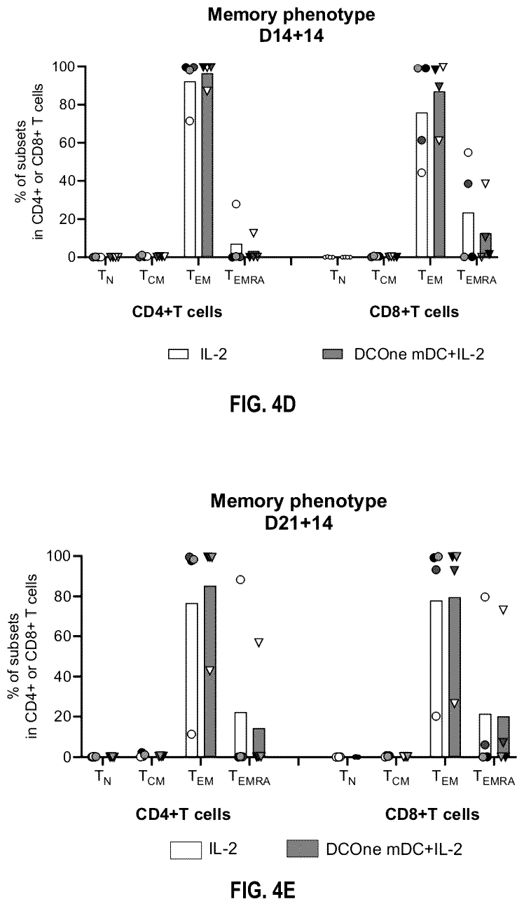

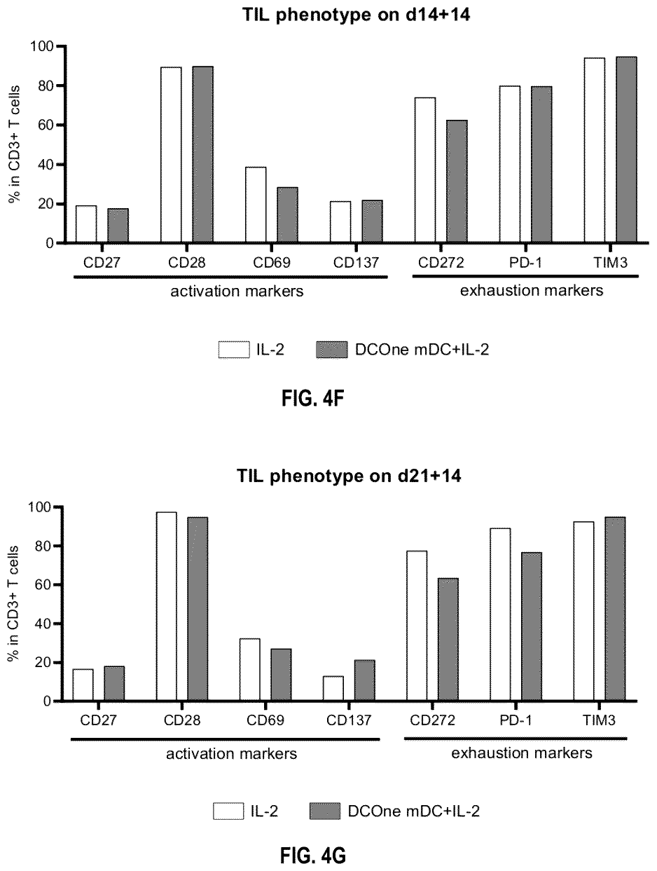

FIGS. 4A and 4B depict the total expansion of TILs from day 14 pre-REP (FIG. 4A) and day 21 (FIG. 4B) pre-REP cultures, with or without addition of DCOne mDCs, subsequently expanded for 14 days using the standard REP-protocol including irradiated PBMC from 3 different healthy donor in the presence of IL-2 (3000 IU/mL) and anti-CD3 antibody (OKT-3 clone).

FIG. 4C depicts the TIL expansion fold obtained in the REP phase, where TILs expanded for 14 days in pre-REP cultures, with or without addition of DCOne mDCs, were subsequently expanded for 14 days using the standard REP-protocol including irradiated PBMC from 3 different healthy donor in the presence of IL-2 (3000 IU/mL) and anti-CD3 antibody (OKT-3 clone).

As shown in 4A, 4B, and 4C, the TILs produced from the pre-REP cultures with addition of DCOne mDCs showed a stronger further expansion (especially CD8+ T cells) during the REP phase (without addition of DCOne mDCs), compared to TILs produced from the standard pre-REP cultures without addition of DCOne mDCs.

FIGS. 4D and 4E depict the frequencies of CD4 and CD8 naive (T_{N}), central memory T_{CM}, effector memory (T_{EM}) and Terminally differentiated effector memory (T_{EMRA} ) T cells in CD4+ and CD8+ T cells in the final REP cultures after TILs from tumor samples were expanded in the pre-REP phase with or without addition of DCOne mDCs for 14 days (FIGS. 4D) and 21 days (FIG. 4E) and subsequently expanded in the REP phase for 14 days.

As shown in FIGS. 4D and 4E, the process of expansion including addition of DCOne mDCs to the standard pre-REP culture resulted in increased frequency of effector memory CD8 T cells during the subsequent REP phase, relative to the process where no DCOne mDCs were added to the standard pre-REP culture. Moreover, it was found that expansion of the TILs previously cultured together with DCOne mDCs in the pre-REP phase led to a significantly lower terminal differentiation into T_{EMRA} cells in the REP phase, compared to TILs previously expanded using the standard pre-REP protocol without addition of DCOne mDCs). FIGS. 4F and 4G depict the expression of activation and inhibition molecules on CD3 T cells in the final REP TIL cultures after TILs from tumor samples were expanded in the pre-REP phase with or without addition of DCOne mDCs for 14 days (FIGS. 4F) and 21 days (FIG. 4G) and subsequently expanded in the standard REP phase without addition of DCOne mDCs for 14 days. As shown in FIGS. 4F and 4G, there was a trend of lower frequency of T cells expressing the exhaustion markers CD272 and PD-1 in the final REP TIL cultures in which TILs were expanded by co-culturing with DCOne mDCs during the pre-REP phase, compared to the final REP TIL cultures in which TILs were expanded by culturing in the standard pre-REP culture without adding DCOne mDCs during the pre-REP phase.

From the investigation in this example, it was surprisingly found that addition of DCOne mDCs to the pre-REP culture significantly increased the expansion of TILs, preferentially expanding CD8+ T cells, during the REP phase (without addition of DCOne mDCs), compared to pre-REP cultures without added DCOne mDCs. Moreover, it was surprisingly found that a strong expansion during the initial pre-REP phase in the presence of DCOne mDCs does not lead to activation-induced exhaustion of the proliferating TILs in the subsequent REP phase, despite an increase in the frequency of more differentiated effector-memory (EM) cells.

### Example 5: CD107a Expression and IFN-gamma Production in Expanded TILs (pre-REP Followed by REP)

TILs from day 14 and day 21 pre-REP cultures, with or without addition of DCOne mDCs, were subsequently expanded for 14 days using the standard REP-protocol in the presence of irradiated PBMC from 3 different healthy donor, IL-2 (3000 IU/mL) and anti-CD3 antibody (OKT-3 clone) for 14 days. At the completion of the REP expansion, TILs from the final cultures were harvested and analyzed for CD107a expression and IFN-gamma (INFγ) production. Phytohemagglutinin (PHA)-induced INFγ production was measured by IFNγELISPOT essay.

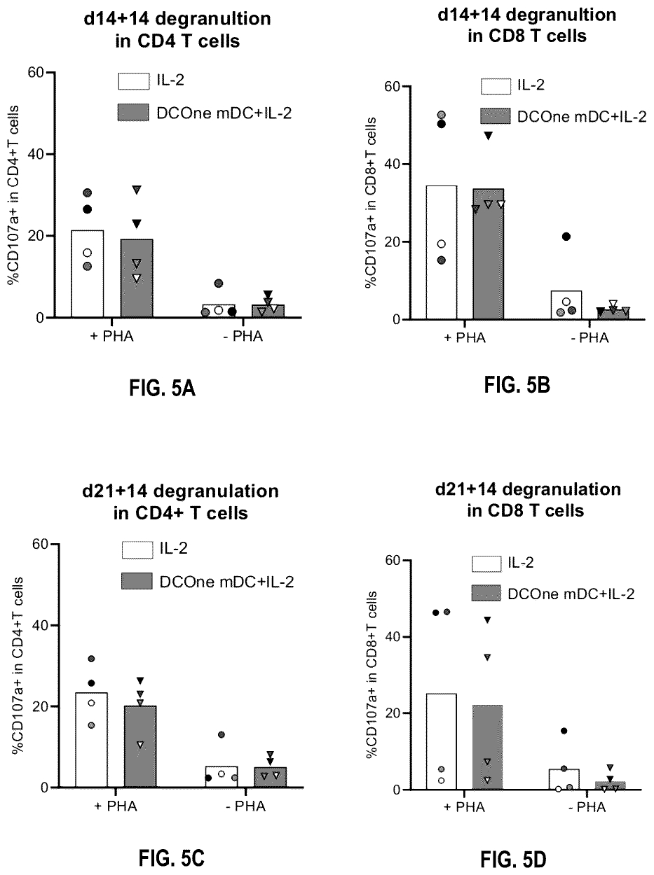

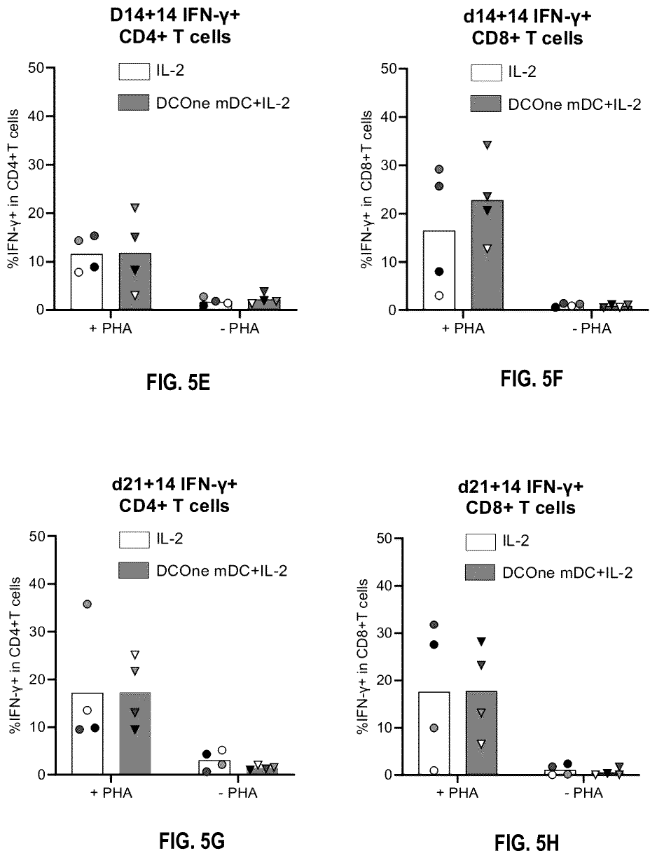

FIGS. 5A-5D depict the frequencies of CD107a expressing CD4+ T cells and CD8+ T cells in the final REP TIL cultures, where the TILs were expanded in the pre-REP phase with or without addition of DCOne mDCs for 14 days (FIG. 5A and FIGS. 5B) and 21 days (FIG. 5C and FIG. 5D) and subsequently expanded in the standard REP phase without addition of DCOne mDCs for 14 days. The results reflect the degranulation capacity (CD107a expression) in CD4+ and CD8+ T cells after stimulation with phytohemagglutinin (PHA). When a cell undergoes degranulation, i.e., releasing cytotoxic granules towards a target cell, the expression of the cell surface protein CD107a is upregulated. Typically, the higher the CD107a expression, the greater the degranulation capacity of the cell. Therefore, CD107a expression assessment can be used to evaluate the degranulation capacity of the expanded TILs.

FIGS. 5E-5H depict the frequencies of CD4+ T cells and CD8+ T cells producing INFγ upon PHA induction in the final TIL cultures, wherein the TILs were expanded in the pre-REP phase with or without addition of DCOne mDCs for 14 days (FIG. 5E and FIGS. 5F) and 21 days (FIG. 5G and FIG. 5H) and subsequently expanded in the standard REP phase without addition of DCOne mDCs for 14 days.

These results show that addition of DCOne mDCs in the pre-REP phase would not negatively affect the frequencies of CD107a expression CD4+ and CD8+ T cells, as well as the capacity of production of INFγ in response to PHA stimulation, in the final REP TIL cultures, relative to the final TIL cultures produced by a process without adding DCOne mDCs in the pre-REP culture. Moreover, it was found that after the TILs were expanded in the pre-REP phase with addition of DCOne mDCs for 14 days and subsequently expanded in the standard REP phase without addition of DCOne mDCs for additional 14 days, the frequency of INFY expressing CD8+ T cells increased, relative to the TILs expanded in the process where no DCOne mDCs were added during the pre-REP phase. The result confirms a lower grade of exhaustion compared to CD8+ T cells from standard cultures without the presence of DCOne mDCs in the pre-REP phase.

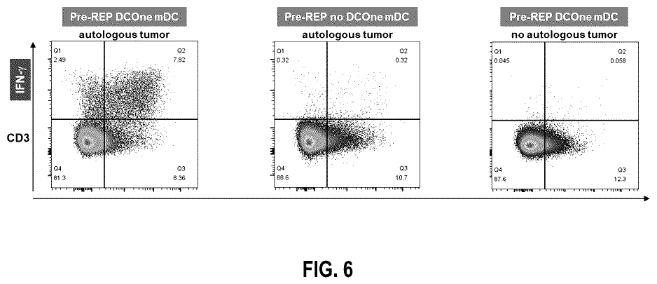

FIG. 6 depicts results of flow cytometry experiments examining IFNγ (y-axis) and CD107 (x-axis) expression levels in CD3+ Tcells upon co-culture with autologous tumor (left and center scatterplots) or no autologous tumor (control, right scatter plot) in the final TIL cultures, wherein the TILs were expanded in the pre-REP phase with (left scatterplot, right scatterplot) or without (center scatterplot) addition of DCOne mDCs for 14 days and subsequently expanded in the standard REP phase without addition of DCOne mDCs for 14 days. Tumor specific CD3+ T cells exhibit higher expression of both IFNγ and CD107 when TIL cultures were expanded in the pre-REP phase with DCOne mDCs (left scatterplot) than without (center scatterplot).

Examples disclosed herein demonstrate that the allogeneic, off-the-shelf, leukemic cell-derived DCs, e.g., DCOne mDCs, can be used to efficiently stimulate and expand TILs from solid tumor tissues ex vivo.

1. A method for ex vivo stimulation and expansion of tumor infiltrating lymphocytes (TILs), comprising:
(a) performing a first phase expansion of TILs by co-culturing a first population of TILs from a tumor sample and a population of modified cells of leukemic origin in a pre-rapid expansion protocol (pre-REP) culture comprising interleukin-2 (IL-2), thereby producing a second population of TILs, wherein the population of modified cells of leukemic origin comprises a mature dendritic cell phenotype and is non-proliferating, and wherein the second population of TILs is greater in number than the first population of TILs; and
(b) performing a second phase of expansion by culturing the second population of TILs in a rapid expansion protocol (REP) culture comprising an anti-CD3 antibody, at least one cytokine, and irradiated allogeneic peripheral blood mononuclear cells (PBMCs) without addition of the population of modified cells of leukemic origin, thereby producing a third population of TILs, wherein the third population of TILs is greater in number than the second population of TILs.

2-7. (canceled)

8. The method of claim 1, wherein the first population of TILs is a population of unselected TILs comprised in the tumor sample, and wherein the tumor sample is co-cultured with the population of modified cells of leukemic origin in the pre-REP culture.

9. The method of claim 1, wherein the tumor sample is from melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma.

10. (canceled)

11. The method of claim 1, wherein the first phase expansion is performed for a period of about 7 days to about 21 days,

12. The method of claim 1, wherein the ratio of the number of the modified cells of leukemic origin added in the first phase expansion culture to the number of TILs in the first population is from about 1:10 to about 1:1.

13. The method of claim 1, wherein IL-2 is present in the pre-REP phase culture at a concentration ranging from about 1000 IU/mL to about 6000 IU/mL.

14-28. (canceled)

29. The method of claim 1, wherein the at least one cytokine present in the culture during the REP phase is selected from IL-2, IL-15, IL-21, and/or IL-7.

30. (canceled)

31. The method of claim 1, wherein the second expansion is performed for a period of up to two weeks,

32-37. (canceled)

38. The method of claim 1, further comprising a step of harvesting and sterilizing the third population of TILs to produce a therapeutic population of TILs suitable for treating a cancer.

39-40. (canceled)

41. A method for expanding tumor infiltrating lymphocytes (TILs) and producing a therapeutic population of TILs for treating cancer, comprising:
(b) obtaining a tumor sample comprising a first population of TILs from a donor subject having cancer;
(b) performing a first expansion of TILs by co-culturing the first population of TILs and a population of modified cells of leukemic origin during a pre-REP phase in a culture comprising interleukin-2 (IL-2) for a period of about 7 days to about 21 days, thereby producing a second population of TILs,
wherein the population of modified cells of leukemic origin comprises a mature dendritic cell phenotype and is non-proliferating, and
wherein the second population of TILs is greater in number than the first population of TILs;
(c) performing a second expansion by culturing the second population of TILs in a REP phase in a culture comprising an anti-CD3 antibody, at least one cytokine, and irradiated allogeneic peripheral blood mononuclear cells (PBMCs) without addition of the population of modified cells of leukemic origin for period of up to 2 weeks, thereby producing a third population of TILs, wherein the third population of TILs is greater in number than the second population of TILs; and
(d) harvesting and sterilizing the third population of TILs to produce a therapeutic population of TILs suitable for treating a cancer in a receiver subject in need thereof.

42. The method of claim 41, wherein the donor subject is the receiver subject, and wherein the therapeutic population of TILs is for autologous treatment.

43-47. (canceled)

48. A method for ex vivo stimulation and expansion of tumor infiltrating lymphocytes (TILs) or enriching CD8+ T cells from a tumor sample, comprising:
performing a pre-rapid expansion of TILs by co-culturing a first population of TILs from a tumor sample and a population of modified cells of leukemic origin in a pre-rapid expansion protocol (pre-REP) culture comprising interleukin-2 (IL-2), thereby producing a second population of TILs,
wherein the population of modified cells of leukemic origin comprises a mature dendritic cell phenotype and is non-proliferating, and
wherein the second population of TILs is greater in number than the first population of TILs.

49. The method of claim 48, wherein the pre-rapid expansion is performed for a period of about 7 days to about 21 days,

50-51. (canceled)

52. The method of claim 48, wherein the first population of TILs is a population of unselected TILs comprised in the tumor sample, and wherein the tumor sample is co-cultured with the population of modified cells of leukemic origin in the pre-REP culture.

53. (canceled)

54. The method of claim 48, wherein the tumor sample is from melanoma, ovarian cancer, endometrial cancer, cervical cancer, non-small-cell lung cancer (NSCLC), lung cancer, bladder cancer, breast cancer, triple negative breast cancer, cancer caused by human papilloma virus, head and neck cancer, head and neck squamous cell carcinoma (HNSCC) renal cancer, and/or renal cell carcinoma.

55-59. (canceled)

60. The method of claim 48, further comprising:
performing a subsequent rapid expansion of TILs by culturing the second population of TILs in a rapid expansion protocol (REP) culture comprising at least one cytokine, without addition of peripheral blood mononuclear cells (PBMCs), thereby producing a third population of TILs, wherein the third population of TILs is greater in number than the second population of TILs.

61. The method of claim 48, further comprising:
performing a subsequent rapid expansion of TILs by culturing the second population of TILs in a rapid expansion protocol (REP) culture comprising at least one cytokine, without addition of any anti-CD3 antibody, thereby producing a third population of TILs, wherein the third population of TILs is greater in number than the second population of TILs.

62. The method of claim 60 wherein the at least one cytokine present in the culture during the REP phase is selected from IL-2, IL-15, IL-21, and/or IL-7.

63. (canceled)

64. The method of claim 60, wherein the rapid expansion is performed for a period of up to two weeks.

65-67. (canceled)

68. The method of claim 61, wherein the at least one cytokine present in the culture during the REP phase is selected from IL-2, IL-15, IL-21, and/or IL-7.
